(19) United States
(12) Patent Application Publication
(10) Pub. No.: US 2024/0141413 A1
(43) Pub. Date: May 2, 2024

(54) AMPLICON-FREE CRISPR-BASED ONE-POT DETECTION WITH LOOP-MEDIATED ISOTHERMAL AMPLIFICATION FOR POINT-OF-CARE DIAGNOSIS OF VIRAL PATHOGENS

(71) Applicant: The Hong Kong University of Science and Technology, Hong Kong (CN)

(72) Inventors: I-Ming HSING, Hong Kong (CN); Yumeng CAO, Hong Kong (CN)

(21) Appl. No.: 18/498,488

(22) Filed: Oct. 31, 2023

Related U.S. Application Data
(60) Provisional application No. 63/421,154, filed on Oct. 31, 2022.

(51) Int. Cl. C12Q 1/6823; C12N 9/22; C12Q 1/44; C12Q 1/6844; C12Q 1/6888; G01N 33/52; G01N 33/543

(52) U.S. Cl. CPC C12Q 1/6823; C12N 9/22; C12Q 1/44; C12Q 1/6844; C12Q 1/6888; G01N 33/52; G01N 33/54326; C12N 2310/10

(57) ABSTRACT

The subject invention relates to a contamination-free method that utilizes CRISPR/Cas enzyme specific sequence recognition towards the loop region in the LAMP amplicons to accomplish one-pot detection for rapid visual readout diagnostics of the presence or absence of target nucleic acid sequences.

### CROSS-REFERENCE TO RELATED APPLICATION

The subject application claims the benefit of U.S. Provisional Application Ser. No. 63/421,154, filed Oct. 31, 2022, which is incorporated herein by reference in its entirety.

### SEQUENCE LISTING

The Sequence Listing for this application is labeled “SeqList-09Jan24.xml” which was created on Jan. 9, 2024 and is 35,914 bytes. The entire contents of the sequence listing is incorporated herein by reference in its entirety.

### FIELD OF THE INVENTION

The present invention is in the field of nucleic acid diagnostics, in particular the contamination-free point-of-care testing diagnostic platforms. This invention involves using AapCas12b to specifically recognize amplified nucleic acid products and destroy one of the loop regions in the product sequence through the rational design of the primer sequence for AapCas12b recognition by sgRNA so that it can eliminate or reduce false positive results in, for example, a non-laboratory setting.

### BACKGROUND OF THE INVENTION

Accurate and rapid point-of-care tests to diagnose patients with an infectious disease will increase the value of the diagnosis outside of a laboratory settings, while also reducing the time to identify positive patients and take proper isolation and clinical treatment.^{1,2} For example, point-of-care tests have important implications, such as in identifying public variants³, on-site testing in airports and hospitals^{4,5}, or even home-based screening in the battle against infectious diseases such as SARS-CoV-2.^{6,7} Currently, qPCR is the gold standard of lab-based virus RNA diagnostics; however, routine qPCR testing suffers the limits of strict contamination control and trained professional must run the tests⁸, which makes it difficult to diagnose on-site tests in a high-risk area. Although there have been improvements in qPCR-based diagnostic tools to shorten turnover time^{8,9}, these require more complex analysis procedures and more specialized personnel. Therefore, more rapid and contamination-free diagnostic methods for point-of-care testing (POCT) of viral pathogens are needed.

CRISPR-based biosensors are good candidates for simple, yet rapid and sensitive point-of-care tests of viral RNA¹⁰. It is usually designed to be triggered by amplified dsDNA products, such as loop-mediated isothermal amplification (LAMP) reaction amplicons. CRISPR/AapCas12b, as a type of RNA-guided endonuclease that reacts at a high temperature around 60° C.¹¹, can be designed to detect a specific target, and upon the cleavage of the target (called “cis cleavage”), unleashes a catalytic, non-specific endonuclease activity on single-stranded DNA (ssDNA) molecules (called “trans cleavage”)¹². As a signal amplification strategy, trans cleavage activity of Cas12b enabled the detection of target molecules as low as sub-attomolar concentrations¹³.

Despite the promising results of CRISPR-based diagnostics, rapid SARA-Cov-2 virus POCT detection methods have limitations: first, the aerosol contamination in a non-laboratory environment from the amplification step can cause high false-positive risk; and second, the trans cleavage can also amplify background signal when activators appear if the initiation step is not well designed. For example, STOP COVID¹¹, is a highly specific and sensitive one-pot assay, but it lacks automation of manual operation protocols, which increases the risk of sample contamination from amplicons. Especially when LAMP, with its high amplification efficiency, is used as the initial step, there will still be many template sequences containing amplicons at the end of the reaction that can be amplified again by LAMP loop primers,¹⁴ even after the CRISPR enzyme cleavage. CRISPR-based one-pot work has also used automatic microfluidics to better control aerosol contamination.¹⁵ Unfortunately, automatic microfluidics need an extra pump instrument, and the POCT prototype costs more than $900, which is unsuitable for at-home test¹⁶. Thus, a novel method of POCT testing using LAMP and CRISPR-based signal transduction is needed.

### BRIEF SUMMARY OF THE INVENTION

The subject invention pertains to novel methods for contamination-free diagnostics by introducing specific sequence loop-mediated isothermal amplification (LAMP) primers for AapCas12b sgRNA recognition as both an activation step and an amplified product cleaning step. In certain embodiments, a PAM code and a specific sequence region for AapCas12b sgRNA recognition is designed in the loop region of LAMP primers. In certain embodiments, the dsDNA products of the LAMP reaction will be recognized by AapCas12b specific cis-cleavage activity and activate its non-canonical trans-cleavage. In preferred embodiments, there will be no amplicons remaining in the one-pot reaction tube ready for LAMP amplification, as the region for loop primer binding is all destroyed by cis cleavage during the AapCas12b activation step.

In certain embodiments, the subject methods pertain to an amplicon-free CRISPR-based one-pot loop-mediated isothermal amplification for point-of-care diagnosis of SARS-CoV-2 RNA and other nucleic acid pathogens. In certain embodiments, AapCas12b sgRNA is designed to recognize the activator sequence, which is designed as the loop region of the LAMP product. In certain embodiments, amplicons of a target nucleic acid sequence, such as, for example, SARS-CoV-2 ssRNA, are cleaved by cis cleavage activity of AapCas12b enzyme until its activation after target amplification.

In certain embodiments, the subject invention can reduce the contamination for point-of-care diagnostics by destroying the amplicon loop sequences at the end of each reaction using CRISPR/Cas enzyme cis cleavage activity that can be hybridized and amplified again with a LAMP primer without the virus RNA target. In certain embodiments, the subject methods can reduce or eliminate false positive results.

Advantageously, the subject invention does require a strict environmental control and professional operators to control contamination. In addition, the subject invention can enhance the sensitivity of one-pot reactions by enhancing the amount of RNA template at the initial stage of amplification.

### BRIEF DESCRIPTION OF THE DRAWINGS

FIG. 1 The reaction mechanism of contamination-free CoLAMP reaction by siting cis cleavage into loop sequence region in LAMP inner primers. Using this design, the CRISPR activation process is then by recognizing LAMP loop structure containing amplicons, without losing any RNA target templates in this step. However, since the cis cleavage site is in loop sequence region, the amplicons can't be amplified again once been recognized and cleaved by CRISPR/Cas. Finally, its collateral cleavage activity will help the signal transduction of signal stranded fluorescence reporter (ssFQ) or double-stranded methylene blue reporter (dsMB) to further produce a visual signal or a digital signal for smartphone users.

FIG. 2 Amplicon-free CRISPR-based one-pot detection mechanism with traditional LAMP primer pool without loop-containing structure initially. Same as the primer pool in LAMP, there are a pair of inner primers, outer primers, and loop primers (FIP is forward inner primer, BIP is backward inner primer, FOP is forward outer primer, BOP is backward outer primer). According to our amplicon-free mechanism, the inner primer is designed by siting a PAM code downstream the target sequence. AapCas12b enzyme sgRNA will be designed for rapid recognition of the sequence before PAM code in this segment, which will be crucial for LAMP exponential amplification based on the LAMP dumbbell structure created in the initial stage. In this way, once there is a target viral pathogen, the target amplification reaction will be transduced to CRISPR signal amplification, amplicons will also be destroyed during this CRISPR activation process before there are too many amplicons (around 10⁹-fold amplicons at the end of traditional LAMP reaction).

FIGS. 3A-3B Results from tests of glycerol additive with 7.5% concentration. (FIG. 3A) Glycerol additive with 7.5% concentration to isolate the CRISPR/Cas mix has significant sensitivity enhancement without sacrificing amplicon-free performance according to SYBR-Green based real-time dsDNA product assessment until 60 mins. (FIG. 3B) Results from the glycerol additive with 7.5% concentration over 60 minutes show that the additive significantly enhanced sensitivity without diminishing amplicon-free performance.

FIGS. 4A-4B The overall point-of-care diagnostic workflow in our design. (FIG. 4A) Our amplicon-free detection can be done within 30 mins from nasopharyngeal swab sampling to signal readout in one tube. And the final visual fluorescence signal can be seen by the naked eye or taken a picture by smartphone. (FIG. 4B) Quantification analysis is easily got from the figure treatment platform RGB measure green channel filtration such as ImageJ analysis.

FIG. 5 The fluorescence-based readout dark chamber. In the case of only a fluorescence filter (525 nm) or only one excitation light source (450 nm), it is impossible to detect the fluorescent signal of a positive control. Because there are too much background light source. For high-quality discrimination readout, a dark environment is designed to make sure the background control is clear as shown in FIG. 3B. Only the combination of a light-emitting diode (LED 450 nm) to excite the fluorophore and a narrow wavelength filter (525 nm) allows to distinguish positive and negative control.

FIG. 6 The sampling flow chamber automatic design utilizes magnetic beads and mineral oil film suitable for point-of-care testing. A flow channel is concluded in our detection for household use, and it is considered to use magnetic beads to transfer the RNA extracted from the virus extraction buffer to the reaction solution and a mineral oil film is used for chamber isolation.

FIGS. 7A-7B The carboxylate magnetic bead SiO₂ surface enhancement of FAM signal. (FIG. 7A) The fluorescence signal has a good linear relationship with the fluorescence concentration under the same concentration of magnetic beads. In the meantime, carboxylate-modified magnetic beans can enhance the fluorescence signal as shown on the right. (FIG. 7B) Under the same concentration of FAM fluorescence label, the fluorescence signal has a good linear relationship with the carboxylate modified magnetic bead amount (Magnetic bead stock concentration: 10 mg/ml). Conversely, we can also obverse a severe inhibition of FAM signal from aminic modified bead due to the excitation wavelength overlap with Fe₃O₄ metal core.

FIG. 8 High incidence angle laser diode light source from the side of the reaction chamber for maximum light reflection enables the best visual signal readout.

FIG. 9 Colormetric readout realized by AuNPs plasmonic surface resonance shifts according to the change of the dispersive states of AuNPs.

FIGS. 10A-10B Lateral flow assay realized by location change of metal NPs (such as AuNPs) by cleaved and uncleaved dual labelled reporter shown as visual red band on chip. (FIG. 10A) Lateral flow assay realized by location change of other colorimetric initiators (such as glucose oxidase) to trigger a downstream colorimetric readout (FIG. 10B).

FIGS. 11A-11B Electrochemical-based readout and signal-on performance. (FIG. 11A) The immobilization-free electrochemical-based readout using methylene blue labeled DNA reporter fragment length identification. (FIG. 11B) Electrochemical signal-on performance with limit of detection (0.5 cps/mL).

FIG. 12 Amplicon-free CRISPR-based one-pot detection mechanism also has good compatibility with loop structure containing LAMP primer pool. Similar to the primer pool in traditional LAMP, there are a pair of inner primers and outer primers (FILP is forward inner loop primer, BILP is backward inner loop primer, FOP is forward outer primer, BOP is backward outer primer). The PAM code is designed within loop-containing LAMP inner primers so that the primer sequence can be flexibly designed without consideration to site a PAM code near primer binding region and to optimize the loop structure conformation process. AapCas12b enzyme sgRNA will be designed for rapid recognition of the sequence in the loop region of FILP (red), which will be crucial for LAMP exponential amplification based on the LAMP dumbbell structure created in initial stage. In this way, once there is a target viral pathogen, the target amplification reaction will be transduced to CRISPR signal amplification, amplicons will also be destroyed during this CRISPR activation process before there is too much amplicons (around 10\hat{ }9 fold amplicons at the end of LAMP reaction).

FIGS. 13A-13E Different rational designs of the sequence of loop structure. (FIG. 13A) Rational design of the sequence of loop structure containing LAMP primer with Pool 5-PAM in loop. (FIG. 13B) Rational design of the sequence of loop structure containing LAMP primer with Pool B-PAM partially in locker. (FIG. 13C) Rational design of the sequence of loop structure containing LAMP primer with Pool 7-PAM in locker. (FIG. 13D) PAM designed in the inner primers locker region can facilitate the unwinding of the locker region of the dumbbell structure created in LAMP initial stage for better sensitivity as well as quicker CRISPR activation leading to better amplicon-free performance in 100 minutes. (FIG. 13E) PAM designed in the inner primers locker region can facilitate the unwinding of the locker region of the dumbbell structure created in LAMP initial stage for better sensitivity as well as quicker CRISPR activation leading to better amplicon-free performance in 60 minutes.

FIGS. 14A-14B Product quantification analysis on loop and result. (FIG. 14A) Product quantification analysis on loop and target region of short products in both positive and negative samples using qPCR. (FIG. 14B) The method of FIG. 14A is shown to be effective and successful.

FIGS. 15A-15B The sgRNA of Confirm-CRISPR/Cas enzyme is designed to check whether there is a sequence from LAMP amplification of a target viral pathogen in CoLAMP product using loop structure containing primers in case of the false positive signal from the introduced sequence on primer loop region primer if they are not well designed. (FIG. 15A) Product sequence characterization reaction by ending-point Confirm-Cas fluorescence signal from SARS-CoV-2. (FIG. 15B) Product sequence characterization reaction by ending-point Confirm-Cas fluorescence signal from the CoLAMP product.

FIGS. 16A-16B Amplicon-free point-of-care diagnostics can be initiated by other typical isothermal amplification method such as RPA. Amplicon-free CRISPR-based one-pot detection mechanism have good compatibility with RPA by designing the recognition sequence partially on the forward or backward primer by siting a PAM code downstream the target for CRISPR/Cas recognition while it is also crucial for exponential amplification. (FIG. 16A) 37° C. CoRPA analytical performance with the PAM-free or PAM-related linear primer design. (FIG. 16B)

FIGS. 17A-17B Loop structure containing primer (named as bubble primer) have good compatibility with RPA by introducing PAM sequence in the stem region upstream the forward primer and backward primer for ultra-sensitive amplification and CRISPR/Cas activation while recognition sequence is sited on target template to take kinetical control of exponential amplification. (FIG. 17A) 37° C. CoRPA analytical performance with the PAM-free or PAM-related bubble primer design. (FIG. 17B)

FIGS. 18A-18B High-conc magnesium acetate (MgOAC) leads to quicker RPA activation at low temperature (30 degree) using both conventional linear primer and loop structure containing primer (named as Bubble primer) design.

### BRIEF DESCRIPTION OF THE SEQUENCES

SEQ ID NO: 1: F3-LAMP forward outer primer to amplify SARS-CoV-2 N Gene

SEQ ID NO: 2: B3-LAMP backward outer primer to amplify SARS-CoV-2 N Gene

SEQ ID NO: 3: LF-LAMP forward loop primer to amplify SARS-CoV-2 N Gene

SEQ ID NO: 4: LB-LAMP backward loop primer to amplify SARS-CoV-2 N Gene

SEQ ID NO: 5: FIP-LAMP forward inner primer to amplify SARS-CoV-2 N Gene

SEQ ID NO: 6: BIP-LAMP backward inner primer to amplify SARS-CoV-2 N Gene

SEQ ID NO: 7: FILP-LAMP forward inner loop primer to amplify SARS-CoV-2 N Gene

SEQ ID NO: 8: BILP-LAMP backward inner loop primer to amplify SARS-CoV-2 N Gene

SEQ ID NO: 9: CRISPR/Cas sgRNA to transduce N gene signal with linear LAMP primer pool in table s2

SEQ ID NO: 10: CRISPR/Cas sgRNA to transduce N gene signal with loop-containing LAMP primer pool in table s3

SEQ ID NO: 11: CRISPR/Cas sgRNA for confirming the LAMP amplicons sequence

SEQ ID NO: 12: FP-1 forward primer to amplify SARS-CoV-2 N gene

SEQ ID NO: 13: FP-2 forward primer to amplify the loop region sequence of the LAMP amplicons from SARS-CoV-2 N gene

SEQ ID NO: 14: BP-1 backward primer to amplify SARS-CoV-2 N gene

SEQ ID NO: 15: dsMB-electrochemical double stranded reporter to transduce signal

SEQ ID NO: 16: dsMB-complementary sequence of the double stranded electrochemical reporter to transduce signal

SEQ ID NO: 17: ssMB-electrochemical single stranded reporter to transduce signal

SEQ ID NO: 18: SARS-CoV-2 N Gene RNA in pseudovirus

SEQ ID NO: 19: Forward primer to amplify SARS-CoV-2 N gene

SEQ ID NO: 20: Backward primer to amplify SARS-CoV-2 N gene

SEQ ID NO: 21: CoRPA crRNA transduce N gene signal with RPA primers

SEQ ID NO: 22: Influenza A M gene Pseudo virus

SEQ ID NO: 23: Forward primer to amplify Influenza A M gene

SEQ ID NO: 24: Backward primer to amplify Influenza A M gene

SEQ ID NO: 25: CoRPA crRNA to transduce M gene signal with RPA primers

SEQ ID NO: 26: HPV 16 E4 cfDNA sequence

SEQ ID NO: 27: Forward primer to amplify HPV 16 E4 cfDNA sequence

SEQ ID NO: 28: Backward primer to amplify HPV 16 E4 cfDNA sequence

SEQ ID NO: 29: Bubble structure containing forward primer to amplify HPV 16 E4 cfDNA sequence

SEQ ID NO: 30: Bubble structure containing backward primer to amplify HPV 16 E4 cfDNA sequence

SEQ ID NO: 31: CoRPA crRNA to transduce N gene signal with PAM-related linear RPA primers

SEQ ID NO: 32: CoRPA crRNA to transduce N gene signal with PAM-free linear RPA primers

SEQ ID NO: 33: CoRPA crRNA to transduce N gene signal with PAM-related bubble RPA primers

SEQ ID NO: 34: CoRPA crRNA to transduce N gene signal with PAM-free bubble RPA primers

### DETAILED DISCLOSURE OF THE INVENTION

The subject invention relates to a contamination-free scheme that utilizes CRISPR/Cas enzyme specific sequence recognition towards the loop region in the LAMP amplicons to accomplish contamination-free, one-pot detection for rapid visual readout SARS-CoV-2 diagnostics in non-lab environments.

In this way, the subject invention can solve the contamination issue for point-of care diagnostics by destroying the amplicons loop sequence at the end of each trail that can be hybridized and amplified again by loop primer without virus RNA target, which is crucial to combat false positive result in negative samples. Advantageously, the subject invention is environmentally-friendly. In addition, the subject invention can solve the sensitivity issue in other one-pot reaction studies that they may lose RNA template at the initial stage of amplification due to the cis cleavage site locating into the target amplification region in their design.

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. The term nucleic acid is used interchangeably with gene, cDNA, and mRNA encoded by a gene.

As used herein, the term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide”, “peptide”, and “protein” are used interchangeably herein to refer to a polymer of amino acids. The terms apply to amino acid polymers in which one or more amino acid residues are artificial chemical mimetic of a corresponding naturally occurring amino acids, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

The term “organism” as used herein includes viruses, bacteria, fungi, plants and animals. Additional examples of organisms are known to a person of ordinary skill in the art and such embodiments are within the purview of the materials and methods disclosed herein. The assays described herein can be useful in analyzing any genetic material obtained from any organism.

The term “genome”, “genomic”, “genetic material” or other grammatical variation thereof as used herein refers to genetic material from any organism. A genetic material can be viral genomic DNA or RNA, nuclear genetic material, such as genomic DNA, or genetic material present in cell organelles, such as mitochondrial DNA or chloroplast DNA. It can also represent the genetic material coming from a natural or artificial mixture or a mixture of genetic material from several organisms.

As used herein, “a target region” or “a target sequence” is a region of interest in genetic material of an organism.

The term “hybridizes with” when used with respect to two sequences indicates that the two sequences are sufficiently complementary to each other to allow nucleotide base pairing between the two sequences. Sequences that hybridize with teach other can be perfectly complementary but can also have mismatches to a certain extent. Therefore, the sequences at the 5′ and 3′ ends of the extension and ligation probes described herein may have a few mismatches with the corresponding target sequences at the 5′ and 3′ ends of the target genomic region as long as the extension and the ligation probes can hybridize with the target sequences to facilitate capturing of the target genomic region. Depending upon the stringency of hybridization, a mismatch of up to about 5% to 20% between the two complementary sequences would allow for hybridization between the two sequences. Typically, high stringency conditions have higher temperature and lower salt concentration and low stringency conditions have lower temperature and higher salt concentration. High stringency conditions for hybridization are preferred, and therefore, the sequences at the 3′ and 5′ ends of the extension and ligation probes are preferred to be perfectly complementary to the corresponding target sequences at the 3′ and 5′ ends of the target genomic region.

Throughout this disclosure, different sequences are described by specific nomenclature, for example, a LAMP primer sequence, PAM code sequence, and target sequence. When such nomenclature is used, it is understood that the identified sequence is substantially identical or substantially reverse complementary to at least a part of the corresponding sequence. For example, “a LAMP primer sequence” describes a sequence that is substantially identical to at least a part of the LAMP primer sequence or substantially reverse complementary to at least a part of the primer LAMP sequence. This is because when a captured target genomic region is converted into a double stranded form comprising the primer binding sequence, the double stranded target genomic region can be sequenced using a primer having a sequence that substantially identical or substantially reverse complementary to at least a part of primer binding sequence. Thus, the nomenclature is used herein to simplify the description of different polynucleotides and parts of polynucleotides used in the methods disclosed here; however, a person of ordinary skill in the art would recognize that appropriate substantially identical or substantially reverse complementary sequences to at least a part of the corresponding sequences could be used to practice the methods disclosed herein.

Also, two sequences that correspond to each other, for example, a target sequence and a primer sequence, have at least 90% sequence identity, preferably, at least 95% sequence identity, even more preferably, at least 97% sequence identify, and most preferably, at least 99% sequence identity, over at least 70%, preferably, at least 80%, even more preferably, at least 90%, and most preferably, at least 95% of the sequences. Alternatively, two sequences that correspond to each other are reverse complementary to each other and have at least 90% perfect matches, preferably, at least 95% perfect matches, even more preferably, at least 97% perfect matches, and most preferably, at least 99% perfect matches in the reverse complementary sequences, over at least 70%, preferably, at least 80%, even more preferably, at least 90%, and most preferably, at least 95% of the sequences. Thus, two sequences that correspond to each other can hybridize with each other or hybridize with a common reference sequence over at least 70%, preferably, at least 80%, even more preferably, at least 90%, and most preferably, at least 95% of the sequences. Preferably, two sequences that correspond to each other are 100% identical over the entire length of the two sequences or 100% reverse complementary over the entire length of the two sequences.

As used herein “CRISPR” refers to a family of DNA sequences found in the genomes of prokaryotic organisms. They are used to detect and destroy DNA in an organism's cells.

As used herein “one pot detection” refers to a method of combining substances where it is all in a single closed tube together.

As used herein “loop-mediated isothermal amplification” (LAMP) is a single-tube technique for the amplification of DNA and a tool to detect certain diseases. LAMP uses about 4 to about 6 primers recognizing about 6 to about 8 distinct regions of target DNA to make a specific amplification reaction.

As used herein “amplicon” is a piece of DNA or RNA that is the source and/or product of amplification and/or replication events.

All references cited herein are hereby incorporated by reference in their entirety.

Compounds for Amplicon-Free CRISPR-Based One-Pot Detection with Loop-Mediated Isothermal Amplification for Point-of-Care Diagnosis of Vital Pathogens

The subject invention provides novel oligonucleotides for amplicon-free CRISPR-based one-pot detection of nucleic acid sequences with LAMP. The subject invention provides a series of at least 4, 5, 6, 7, 8, 9, 10 or more LAMP primers that can be used in the subject methods. In certain embodiments, the at least 4 primers comprise a forward outer primer, forward inner primer, backward outer primer, and backward inner primer. In certain embodiments, each primer can hybridize to target sequence, in which the target sequence is derived from genetic material from an organism or virus of interest, such as, for example, SARS-CoV-2.

In certain embodiments, the LAMP primer can encode a site for small guide RNA (sgRNA) recognition, preferably in a loop region that results from the hybridization and extension of a forward inner or backward inner primer. In certain embodiments, the LAMP primers of the subject invention can comprise a specific sequence for AapCas12b sgRNA recognition as both an activation step and an amplified product cleaning step. In certain embodiments, the LAMP forward or backward inner primer can be designed with a PAM code about 40 bases downstream of the sequence that is complementary to the target sequence. In certain embodiments, the PAM code sequence is immediately downstream of the complementary sequence or at least 1, 2, 3, 4, 5, 6, 7, 8, 9, 10 or more nucleotides downstream of the complementary sequence. In certain embodiments, the sgRNA of Confirm-CRISPR/Cas enzyme is designed to check whether there is a sequence from LAMP amplification of a target viral pathogen in CoLAMP product using loop structure containing primers to make sure the CRISPR/Cas activator is from a specific amplification. In certain embodiments, the amplification reaction can be transduced to a CRISPR signal amplification. In certain embodiments, CRISPR/Cas can be activated by specific cis cleavage activity on the target DNA and then CRISPR/Cas can cleave the surrounding single stranded DNA that serve as reporters.

In certain embodiments, the AapCas12b enzyme sgRNA recognizes and hybridizes to the pair chain containing the complementary sequence of primer loop region after the target amplification, which will become the loop region of the LAMP dumbbell structure. In certain embodiments, the Cas12a enzyme can use the sgRNA recognition site on the primer sequence that is exponentially amplified after the target binding initiation stage. In certain embodiments, the recognition site of the sgRNA can be different than the primer binding site. In certain embodiments, the recognition site of the sgRNA can be a sequence complementary with the target nucleotide sequence and about 20 bases downstream of the forward or backward primer, which is within the loop region in the dumbbell structure. In certain embodiments, a PAM code is about 40 bases downstream of the target for CRISPR/Cas recognition.

In certain embodiments, the CRISPR recognition sequence can be designed in recombinase polymerase amplification using a PAM-free seed region within about 10 bases downstream the target sequence or by modifying a TTN PAM code in the stem region (7 bp) of a loop containing primer for CRISPR/Cas recognition.

In certain embodiments, the amplification of the target sequence can use probes to identify and/or quantify the amplified target sequence. In certain embodiments, the probes can produce a signal generated from a single-stranded and/or double-stranded DNA reporter, such as, for example, a single-stranded DNA fluorescence reporter (ssFQ). In certain embodiments, probes can be used to produce a signal generated from a double or single-stranded DNA methylene blue reporter (dsMB/ssMB). In certain embodiments, the fluorophore is at the 3′ end and quencher is at the 5′ end respectively with a 5 unit single stranded DNA sequence, such as, for example, TTTTT, which is optimized to achieve highest signal in the middle. In other embodiments, the fluorescence signal is generated from excitation of cleaved fluorophore labelled reporters. Further, the simple visual readout is realized by an optical filter on the top of the dark chamber to filtrate the extra lights with a wavelength other than that of the emission light (520 nm to 530 nm) to distinguish positive signal (green color) and negative (dark background color).

In certain embodiments, fluorophores are used as labels to generate a fluorescently labeled probe included in embodiments of methods and compositions of the present invention can be any of numerous fluorophores including, but not limited to, 4-acetamido-4′-isothiocyanatostilbene-2,2′ disulfonic acid; acridine and derivatives such as acridine and acridine isothiocyanate; 4-amino-N-[3-vinylsulfonyl)phenyl]naphthalimide-3,5 disulfonate, Lucifer Yellow VS; N-(4-anilino-1-naphthyl)maleimide; anthranilamide, Brilliant Yellow; BIODIPY fluorophores (4,4-difluoro-4-bora-3a,4a-diaza-s-indacenes); coumarin and derivatives such as coumarin, 7-amino-4-methylcoumarin (AMC, Coumarin 120), 7-amino-4-trifluoromethylcoumarin (Coumarin 151); cyanosine; DAPDXYL sulfonyl chloride; 4′,6-diaminidino-2-phenylindole (DAPI); 5′,5″-dibromopyrogallol-sulfonephthalein (Bromopyrogallol Red); 7-di ethyl amino-3-(4′-isothiocyanatophenyl)-4-methyl coumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-4′-dimethylaminophenylazo)benzoic acid (DABCYL); 4-dimethylaminophenylazophenyl-4′-isothiocyanate (DABITC); EDANS (5-[(2-aminoethyl)amino]naphthalene-1-sulfonic acid), eosin and derivatives such as eosin isothiocyanate; erythrosin and derivatives such as erythrosin B and erythrosin isothiocyanate; ethidium such as ethidium bromide; fluorescein and derivatives such as 5-carboxyfluorescein (FAM), hexachlorofluorescenin (HEX), 5-(4,6-dichlorotriazin-2-yl)aminofluorescein (DTAF), 2′,7′-dimethoxy-4′,5′-dichloro-6-carboxyfluorescein (JOE) and fluorescein isothiocyanate (FITC); fluorescamine; green fluorescent protein and derivatives such as EBFP, EBFP2, ECFP, and YFP; IAEDANS (5-({2-[(iodoacetyl)amino]ethyl} amino)naphthalene-1-sulfonic acid), Malachite Green isothiocyanate; 4-methylumbelliferone; orthocresolphthalein; nitrotyrosine; pararosaniline; Phenol Red; B-phycoerytnin; o-phthaldialdehyde; pyrene and derivatives such as pyrene butyrate, 1-pyrenesulfonyl chloride and succinimidyl 1-pyrene butyrate; QSY 7; QSY 9; Reactive Red 4 (Cibacron® Brilliant Red 3B-A); rhodamine and derivatives such as 6-carboxy-X-rhodamine (ROX), 6-carboxyrhodamine (Rhodamine 6G), rhodamine isothiocyanate, lissamine rhodamine B sulfonyl chloride, rhodamine B, rhodamine 123, sulforhodamine B, sulforhodamine 101 and sulfonyl chloride derivative of sulforhodamine 101 (Texas Red); N,N,N′,N-tetramethyl-carboxyrhodamine (TAMRA); tetramethyl rhodamine; tetramethyl rhodamine isothiocyanate (TRITC); riboflavin; rosolic acid; and terbium chelate derivatives.

In certain embodiments, the fluorescent moiety can comprise a fluorescent protein, such as, for example, a green fluorescent protein (GFP); a modified derivative of GFP, including, for example, a GFP comprising S65T, an enhanced GFP; blue fluorescent protein, including, for example, EBFP, EBFP2, Azurite, and mKalamal; cyan fluorescent protein, such as, for example, ECFP, Cerulean, CyPet, mTurquoise2; and yellow fluorescent protein derivatives such as, for example, YFP, Citrine, Venus, YPet.

Exemplary quencher labels include a fluorophore, a quantum dot, a metal nanoparticle, and other related labels. Suitable quenchers include Black Hole Quencher®-1 (Biosearch Technologies, Novato, CA), BHQ-2, Dabcyl, Iowa Black® FQ (Integrated DNA Technologies, Coralville, IA), IowaBlack RQ, QXL™ (AnaSpec, Fremont, CA), QSY 7, QSY 9, QSY 21, QSY 35, IRDye QC, BBQ-650, Atto 540Q, Atto 575Q, Atto 575Q, MGB 3′ CDPI3, and MGB-5′ CDPI3. In one instance, the term “quencher” refers to a substance which reduces emission from a fluorescent donor when in proximity to the donor. In preferred embodiments, the quencher is within 10, 11, 12, 13, 14, 15, 16, 17, 18, 19, 20, 21, 22, 23, 24, 25, 26, 27, 28, 29 or 30 nucleotide bases of the fluorescent label. Fluorescence is quenched when the fluorescence emitted from the fluorophore is detectably reduced, such as reduced by 10%, 20%, 30%, 40%, 50%, 60%, 70%, 80%, 90%, 95%, 99% or more.

In certain embodiments, the concentration of the fluorescent probe in the compositions and method of use is about 0.01 μM to about 1000 μM, about 0.1 μM to about 100 μM, about 0.1 μM to about 50 μM, about 0.1 μM to about 10 μM, or about 0.5 μM to about 3 μM. In certain embodiments, the concentration of the fluorescent probe is about 0.01 μM, about 0.1 μM, 0.2 μM, about 0.25 μM, about 0.3 μM, about 0.4 μM, about 0.5 μM, about 0.6 μM, about 0.7 μM, about 0.8 μM, about 0.9 μM, about 1 μM, about 1.5 μM, about 2 μM, about 2.5 μM, or about 3 μM.

In certain embodiments, dyes ranging between 500 nm to 700 nm have the advantage of being in the visible spectrum and can be detected using existing photomultiplier tubes. In some embodiments, the broad range of available dyes allows selection of dye sets that have emission wavelengths that are spread across the detection range. Detection systems capable of distinguishing many dyes are known in the art. In some embodiments, the visual fluorescence signal can be seen by the naked eye or taken a picture of by smartphone.

In certain embodiments, the composition of the subject invention can contain other compounds, such as fluorophores, oligonucleotides, preservatives, buffers, and any combination thereof. These compounds can be added to the composition can be included in the composition at 0.01 to 99.9%, 0.1 to 90%, 0.5 to 80%, 0.75 to 70%, 1.0 to 50%, 1.5 to 25%, or 2.0 to 15% by weight, with respect to the total composition.

The subject invention also relates to detection kits and diagnostic kits. The kits include oligonucleotide probes and primers, packaged into suitable packaging material, optionally in combination with instructions for using the kit components, e.g., instructions for performing a method of the invention. In one embodiment, a kit includes an amount of an oligonucleotide's probes and primers, and instructions for running the assay on a label or packaging insert. In further embodiments, a kit includes an article of manufacture, for performing the assay. Preferably, said kit comprises at one primer pair according to the invention. Said kit comprises more than one probe, e.g., at least two, at least three, at least four, at least five, at least six, at least 7, at least 8, at least 9, or at least 10 different probes, notably when the kit is intended to discriminate between different SARS-CoV-2 types or other infectious agents or genetic variations that cause disease.

In the kit according to the invention, the oligonucleotides (primers, probes) can be either kept separately, or partially mixed, or totally mixed. In a preferred embodiment, the kit according to the invention can also contain further reagents suitable for a PCR or RT-PCR step.

Such reagents are known to those skilled in the art, and include water, like nuclease-free water, RNase free water, DNAse-free water, PCR-grade water; salts, like magnesium, magnesium chloride, potassium; buffers such as Tris; enzymes, including polymerases, such as Taq, Vent, Pfu (all of them Trade-Marks), activatable polymerase, reverse transcriptase, and the like; nucleotides like deoxynucleotides, dideoxunucleotides, dNTPs, dATP, dTTP, dCTP, dGTP, dUTP; other reagents, like DTT and/or RNase inhibitors; and polynucleotides like polyT, polydT, and other oligonucleotides, e.g., primers.

In another preferred embodiment, the kit according to the invention comprises PCR controls. Such controls are known in the art, and include qualitative controls, positive controls, negative controls, internal controls, quantitative controls, internal quantitative controls, as well as calibration ranges. The internal control for said PCR step can be a template which is unrelated to the target template in the PCR step. Such controls also may comprise control primers and/or control probes. For example, in the case of SARS-CoV-2 detection, it is possible to use as an internal control, a polynucleotide chosen within a gene whose presence is excluded in a sample originating from a human body (for example, from a plant gene), and whose size and GC content is equivalent to those from the target sequence. In other embodiments, a positive control is included which comprises a polynucleotide sequence associated with the target nucleotide sequence, such as an unmutated portion of the target nucleotide sequence (or amplicon). In some embodiments, the positive control is amplified by the oligonucleotide primer pair used to amplify the target nucleic acid sequence. By way of example, positive control sequences for SARS-CoV-2 can be portions of the envelope, membrane, nucleocapsid gene, or invariant regions of the gene encoding the spike protein. For cells derived from specific human tissues, the positive control could be, for example, a portion of the following: the beta-actin gene, the aldolase gene, the dihydrofolate reductase gene, the glyceraldehyde phosphate dehydrogenase gene, the histone 3.3 gene, the hypoxanthine phosphoribosyltransferase gene, the Abelson gene (ABI), the BCR gene, the porphobilinogen deaminase gene (PBGD), or the beta-2-microglobulin gene (12-MG).

In a preferred embodiment, the kit according to the invention contains means for extracting and/or purifying nucleic acid from a biological sample, e.g., from blood, serum, plasma, saliva, or nasal secretions. Such means are well known to those skilled in the art.

In a preferred embodiment, the kit according to the invention contains instructions for the use thereof. Said instructions can advantageously be a leaflet, a card, or the like. Said instructions can also be present under two forms: a detailed one, gathering exhaustive information about the kit and the use thereof, possibly also including literature data; and a quick-guide form or a memo, e.g., in the shape of a card, gathering the essential information needed for the use thereof. Instructions can therefore include instructions for practicing any of the methods of the invention described herein. For example, compositions can be included in a container, pack, or dispenser together with instructions for performing the nucleotide detection assay. Instructions may additionally include storage information, expiration date, or any information required by regulatory agencies such as the Food and Drug Administration or European Medicines Agency for use with a human or animal subject. The instructions may be on “printed matter,” e.g., on paper or cardboard within the kit, on a label affixed to the kit or packaging material or attached to a vial or tube containing a component of the kit. Instructions may comprise voice or video tape and additionally be included on a computer readable medium, such as a disk (floppy diskette or hard disk), optical CD such as CD- or DVD-ROM/RAM, magnetic tape, flash storage, electrical storage media such as RAM and ROM and hybrids of these such as magnetic/optical storage media.

In a preferred embodiment, said kit is a diagnostics kit, especially an in vitro diagnostics kit, i.e., a SARS-CoV-2 diagnostics kit.

TABLE 1
Sequence Table

| No. | Label | Sequence (5′to3′) | units |
|---|---|---|---|
| SEQ ID | F3 | GCTGCTGAGGCTTCTAAG | 18 |
| NO: 1 | | | |
| SEQ ID | B3 | GCGTCAATATGCTTATTCAGC | 21 |
| NO: 2 | | | |
| SEQ ID | LF | CCTTGTCTGATTAGTTCCTGGT | 22 |
| NO: 3 | | | |
| SEQ ID | LB | TGGCATGGAAGTCACACC | 18 |
| NO: 4 | | | |
| SEQ ID | FIP | GCGGCCAATGTTTGTAATCAGTAGACGTGGTC | 40 |
| NO: 5 | | CAGAACAA | |
| SEQ ID | BIP | TCAGCGTTCTTCGGAATGTCGCTGTGTAGGTC | 39 |
| NO: 6 | | AACCACG | |
| SEQ ID | FILP | GGCCTTCAACTCCAACTTCCAACTAAATACGC | 62 |
| NO: 7 | | CCATCGAAGGCCAGACGTGGTCCAGAACAA | |
| SEQ ID | BILP | GGCTCGGGCCCACTCCTACTAAACTCCCACTC | 62 |
| NO: 8 | | ACTAACCGAGCCCTGTGTAGGTCAACCACG | |
| SEQ ID | Co liner- | GUCUAGAGGACAGAAUUUUUCAACGGGUGU | 111 |
| NO: 9 | sgRNA | GCCAAUGGCCACUUUCCAGGUGGCAAAGCCC | |
| | | GUUGAGCUUCUCAAAUCUGAGAAGUGGCAC | |
| | | GGAAUGUCGCGCAUUGGCAU | |
| SEQ ID | Co loop-sgRNA | GUCUAGAGGACAGAAUUUUUCAACGGGUGU | 111 |
| NO: 10 | | GCCAAUGGCCACUUUCCAGGUGGCAAAGCCC | |
| | | GUUGAGCUUCUCAAAUCUGAGAAGUGGCAC | |
| | | AACTCCAACTTCCAACTAAA | |
| SEQ ID | Co loop- | GUCUAGAGGACAGAAUUUUUCAACGGGUGU | 111 |
| NO: 11 | confirm-sgRNA | GCCAAUGGCCACUUUCCAGGUGGCAAAGCCC | |
| | | GUUGAGCUUCUCAAAUCUGAGAAGUGGCAC | |
| | | AGCGCUGGGGGCAAAUUGUG | |
| SEQ ID | FP-1 | AGACGTGGTCCAGAACAA | 18 |
| NO: 12 | | | |
| SEQ ID | FP-2 | TTTAGTTGGAAGTTGGAGTT | 20 |
| NO: 13 | | | |
| SEQ ID | BP-1 | CTGTGTAGGTCAACCACG | 18 |
| NO: 14 | | | |
| | ssFQ-HEX | 5′FAM-TTTTT-3′BHQ1 | 5 |
| | ssFQ-FAM | 5′HEX-TTTTT-3′BHQ1 | 5 |
| SEQ ID | dsMB | 5′MB- | 60 |
| NO: 15 | | TTTTTAAAAAAAAAAAAAAAAAAAAAAA | |
| | | AAAAAAAAAAAAAAAAAAAAAAAAAAATTT | |
| | | TT-3′MB | |
| SEQ ID | dsMB-c | 5′MB-AAAAATTTTTTTTTTTTTTTTTTTTTTTTT | 60 |
| NO: 16 | | TTTTTTTTTTTTTTTTTTTTTTTTTAAAAA-3′MB | |
| SEQ ID | ssMB | 5′MB-TTTTTTTTTTTTTTTTTTTTTTTTTTTTTT | 60 |
| NO: 17 | | TTTTTTTTTTTTTTTTTTTTTTTTTTTTTT-3′MB | |
| SEQ ID | SARS-CoV-2 N | AUGUCUGAUAAUGGACCCCAAAAUCAGCGA | 1261 |
| NO: 18 | Gene in Pseudo | AAUGCACCCCGCAUUACGUUUGGUGGACCCU | |
| | virus | CAGAUUCAACUGGCAGUAACCAGAAUGGAG | |
| | | AACGCAGUGGGGCGCGAUCAAAACAACGUC | |
| | | GGCCCCAAGGUUUACCCAAUAAUACUGCGUC | |
| | | UUGGUUCACCGCUCUCACUCAACAUGGCAAG | |
| | | GAAGACCUUAAAUUCCCUCGAGGACAAGGC | |
| | | GUUCCAAUUAACACCAAUAGCAGUCCAGAU | |
| | | GACCAAAUUGGCUACUACCGAAGAGCUACCA | |
| | | GACGAAUUCGUGGUGGUGACGGUAAAAUGA | |
| | | AAGAUCUCAGUCCAAGAUGGUAUUUCUACU | |
| | | ACCUAGGAACUGGGCCAGAAGCUGGACUUCC | |
| | | CUAUGGUGCUAACAAAGACGGCAUCAUAUG | |
| | | GGUUGCAACUGAUGGGAGCCUUGAAUACAC | |
| | | CAAAAGAUCACAUUGGCACCCGCAAUCCUGC | |
| | | UAACAAUGCUGCAAUCGUGCUACAACUUCCU | |
| | | CAAGGAACAACAUUGCCAAAAGGCUUCUAC | |
| | | GCAGAAGGGAGCAGAGGCGGCAGUCAAGCC | |
| | | UCUUCUCGUUCCUCAUCACGUAGUCGCAACA | |
| | | GUUCAAGAAAUUCAACUCCAGGCAGCAGUA | |
| | | GGGGAACUUCUCCUGCUAGAAUGGCUGGCA | |
| | | AUGGCGGUGAUGCUGCUCUUGCUUUGCUGC | |
| | | UGCUUGACAGAUUGAACCAGCUUGAGAGCA | |
| | | AAAUGUCUGGUAAAGGCCAACAACAACAAG | |
| | | GCCAAACUGUCACUAAGAAAUCUGCUGCUG | |
| | | AGGCUUCUAAGAAGCCUCGGCAAAAACGUA | |
| | | CUGCCACUAAAGCAUACAAUGUAACACAAGC | |
| | | UUUCGGCAGACGUGGUCCAGAACAAACCCAA | |
| | | GGAAAUUUUGGGGACCAGGAACUAAUCAGA | |
| | | CAAGGAACUGAUUACAAACAUUGGCCGCAA | |
| | | AUUGCACAAUUUGCCCCCAGCGCUUCAGCGU | |
| | | UCUUCGGAAUGUCGCGCAUUGGCAUGGAAG | |
| | | UCACACCUUCGGGAACGUGGUUGACCUACAC | |
| | | AGGUGCCAUCAAAUUGGAUGACAAAGAUCC | |
| | | AAAUUUCAAAGAUCAAGUCAUUUUGCUGAA | |
| | | UAAGCAUAUUGACGCAUACAAAACAUUCCC | |
| | | ACCAACAGAGCCUAAAAAGGACAAAAAGAA | |
| | | GAAGGCUGAUGAAACUCAAGCCUUACCGCA | |
| | | GAGACAGAAGAAACAGCAAACUGUGACUCU | |
| | | UCUUCCUGCUGCAGAUUUGGAUGAUUUCUC | |
| | | CAAACAAUUGCAACAAUCCAUGAGCAGUGC | |
| | | UGACUCAACUCAGGCCUAA | |
| SEQ ID | N gene-FP | CCAAGGTTTACCCAATAATACTGCGTCT | 28 |
| NO: 19 | | | |
| SEQ ID | N gene-BP | TCTTTCATTTTACCGTCACCACCACGA | 27 |
| NO: 20 | | | |
| SEQ ID | N gene CoRPA | UAAUUUCUACUAAGUGUAGAUAUUUUACCG | 41 |
| NO: 21 | crRNA | UCACCACCACG | |
| SEQ ID | Infuenza A M | TAGATGTTTAAAGATGAGTCTTCTAACCGAGG | 269 |
| NO: 22 | gene Pseudo | TCGAAACGTACGTTCTTTCTATCATACCGTCA | |
| | virus | GGCCCCCTCAAAGCCGAGATTGCGCAGAGAC | |
| | | TGGAAAGTGTCTTTGCAGGAAAGAATACAGA | |
| | | CCTTGAGGCTCTCATGGAATGGCTAAAGACA | |
| | | AGACCAATCTTGTCACCTTTGACTAAGGGAAT | |
| | | TTTAGGATTTGTATTCACGCTCACCGTGCCCA | |
| | | GTGAGCGAGGACTGCAGCGTAGACGCTTTGT | |
| | | CCAAAATGCCCTGAATG | |
| SEQ ID | M gene-FP | ACCGAGGTCGAAACGTATGTTCTCTCTATC | 30 |
| NO: 23 | | | |
| SEQ ID | M gene-BP | TCTACGCTGCAGTCCTCGCTCACTGGGCAC | 30 |
| NO: 24 | | | |
| SEQ ID | M gene CoRPA | UAAUUUCUACUAAGUGUAGAUAGGGGGCCU | 41 |
| NO: 25 | crRNA | GACGGUAUGAU | |
| SEQ ID | HPV 16 E4 | TATTATGTCCTACATCTGTGTTTAGCAGCGAC | 288 |
| NO: 26 | cfDNA | GAAGTATCCTCTGCTGAAATTATTAGGCAGCA | |
| | sequence | CTTGGCCAACCACTCCGCCGCGACCCATCCCA | |
| | | AAGCCGTCGCCTTGGGCACCAAAGAATCACA | |
| | | GACGACTATCCAGCGACCAAGATCAGAGCCA | |
| | | GACACCGGAAACCCCTGCCACACCAATAAGT | |
| | | TGTTGCACAGAGACTCAGTGGACAGTGCTCCA | |
| | | ATTCTCACTGCAGTTAACAGCTCACACAAAGC | |
| | | ACGGATTAACTGTAATAGTAACACTACACCCA | |
| | | TAG | |
| SEQ ID | HPV16E4 FP | AAGTATCCTCTGCTGAAATTATTAGGC | 27 |
| NO: 27 | | | |
| SEQ ID | HPV16E4 BP | CTATTACAGTTAATCCGTGCTTTGTGTG | 28 |
| NO: 28 | | | |
| SEQ ID | HPV16E4 | AGATTTCGAAGGTGTGAGGGTGGGAGAGAAA | 61 |
| NO: 29 | Bubble FP | TCTAAGTATCCTCTGCTGAAATTATTAGGC | |
| SEQ ID | HPV16E4 | AGATTTCGAAGGTGTGAGGGTGGGAGAGAAA | 62 |
| NO: 30 | Bubble BP | TCTCTATTACAGTTAATCCGTGCTTTGTGTG | |
| SEQ ID | HPV16E4 | UAAUUUCUACUAAGUGUAGAUAGAGGAUAC | 41 |
| NO: 31 | CORPA crRNA | UUCGUCGCUGC | |
| | PAM-related-1 | | |
| SEQ ID | HPV16E4 | UAAUUUCUACUAAGUGUAGAUGCCAAGUGC | 41 |
| NO: 32 | CORPA crRNA | UGCCUAAUAAU | |
| | PAM-free-1 | | |
| SEQ ID | HPV16E4 | UAAUUUCUACUAAGUGUAGAUCAGUUAAUC | 41 |
| NO: 33 | CORPA crRNA | CGUGCUUUGUG | |
| | PAM-related-2 | | |
| SEQ ID | HPV16E4 | UAAUUUCUACUAAGUGUAGAUGAAGUAUCC | 41 |
| NO: 34 | CORPA crRNA | UCUGCUGAAAU | |
| | PAM-free-2 | | |

TABLE 2
Linear CoLAMP primer pool (10X)

| component | Initial conc | Final conc | Amount(μl) |
|---|---|---|---|
| ZF-FIP | 100 uM | 8 uM | 0.4 |
| ZF-BIP | 100 uM | 8 uM | 0.4 |
| ZF-F3 | 100 uM | 1 uM | 0.05 |
| ZF-B3 | 100 uM | 1 uM | 0.05 |
| ZF-LF | 100 uM | 2 uM | 0.1 |
| ZF-LB | 100 uM | 2 uM | 0.1 |
| ddH2O | — | — | 3.9 |
| Total | — | — | 5 |

TABLE 3
Loop-containing CoLAMP primer pool (10X)

| cmponent | Initial conc | Final conc | Amount(μl) |
|---|---|---|---|
| FILP | 100 uM | 8 uM | 0.4 |
| BILP | 100 uM | 8 uM | 0.4 |
| ZF-F3 | 100 uM | 1 uM | 0.05 |
| ZF-B3 | 100 uM | 1 uM | 0.05 |
| ddH2O | — | — | 4.1 |
| Total | — | — | 5 |

Methods for Amplicon-Free CRISPR-Based One-Pot Detection with Loop-Mediated Isothermal Amplification for Point-of-Care Diagnosis of Vital Pathogens

### List of Steps of CoLAMP Assay

In certain embodiments, a swab is placed into the extraction chamber in an extraction buffer for about 5 minutes at about room temperature. In certain embodiments, a sample is injected into a reaction chamber in a CoLAMP mix (Table 4) for 30 min at about 60° C. In certain embodiments, a visual readout is observed in a CoLAMP mix (Table 4) for about 1 min at about room temperature.

In certain embodiments, when performing electrochemical readout, a methylene-blue modified electrochemical reporter (ssMB) can be used instead of Reporter ssDNA (5′FAM-TTTTT-3′BHQ1) at a final concentration of 800 nM titrated for the best signal-to-noise ratio. In addition, a HEX fluorescence label can be utilized instead of FAM (ssHEX reporter in Table 1) in an optimization process to check both CRISPR/Cas cleavage activity and amplicon accumulation. If the target is DNA instead of RNA, the Rtx Reverse Transcriptase can be omitted from the mix buffer.

List of Steps of CoRPA Assay

In certain embodiments, a swab is placed into the extraction chamber in an extraction buffer for about 5 minutes at about room temperature. In certain embodiments, a sample is injected into a reaction chamber in a CoRPA mix (Table 5) for 30 min at about 30 to about 37° C. In certain embodiments, a visual readout is observed in a CoRPA mix (Table 5) for about 1 min at about room temperature.

In certain embodiments, when performing electrochemical readout, a methylene-blue modified electrochemical reporter (ssMB) can be used instead of HPV16 crRNA-v2 at a final concentration of about 800 nM titrated for the best signal-to-noise ratio. In addition, a HEX fluorescence label can be utilized instead of FAM (ssHEX reporter in Table 1) in an optimization process to check both CRISPR/Cas cleavage activity and amplicon accumulation. If the target is DNA instead of RNA, the RT Script IV can be omitted from the CoRPA mix buffer.

TABLE 4
CoLAMP mix buffer

| | | | |
|---|---|---|---|
| Component | Initial conc | Final conc | Amount(μl) |

CoLAMP mix buffer

| Tris-HCl | 1000 | mM | 20 | mM | 1 |
|---|---|---|---|---|---|
| (NH4)2SO4 | 2000 | mM | 10 | mM | 0.25 |
| Tween 20 | 100% | | 0.1% | | 0.05 |
| Nuclease free H2O | — | | — | | 4.2 |
| dNTPs | 10 | mM | 1.4 | mM | 7 |
| MgSO4 | 100 | mM | 8 | mM | 4 |
| Bst 2.0 DNA polymerase | 8,000 | units/mL | 320 | units/mL | 2 |
| Rtx Reverse Transcriptase | 15,000 | units/mL | 300 | units/mL | 1 |
| Taurine | 500 | mM | 50 | mM | 5 |

| | | | |
|---|---|---|---|
| Proteinase K inhibitor | 10X | 1X | 5 |
| SYBR green | 100X | 1X | 0.5 |
| 10X LAMP primer pool | 10X | 1X | 5 |

AapCas12b mix

| | | | | | |
|---|---|---|---|---|---|
| AapCas12b protein | 2 | uM | 31.25 | nM | 0.781 |
| AapCas12b sgRNA | 10 | uM | 31.25 | nM | 0.156 |
| Reporter ssDNA | 10 | uM | 250 | nM | 1.25 |

| | | | |
|---|---|---|---|
| (5′FAM -TTTTT-3′BHQ1)* | | | |
| glycerol. | 100% | — | 3.8 |
| Nuclease-free water | — | — | 14.313 |
| N gene RNA | — | — | — |
| Total | | | 50 |

TABLE 5
CoRPA mix buffer

| | | | |
|---|---|---|---|
| Component | Initial conc | Final conc | Amount(μl) |

CoRPA mix buffer

| RT Script IV | 200 | U/μL | 100 U (10-200 U) | | 0.5 |
|---|---|---|---|---|---|
| RNase inhibitor | 40 | U/μl | 40 U (20-200 U) | | 1 |
| RPA buffer | 24.4 | | | | |
| Primer pool | 10 | μM | 0.3125 | μM | 2.5 |
| Rehydration buffer | — | | — | | 16 |
| MgOAC (20X) | 280 | mM | 10 | mM | 1.43 |
| Syto 16 (50 μM) | 10 x | | 1X | (5 μM) | 4 |
| dNTP | 10 | mM | 1 | mM | 4 |
| ATP | 30 | mM | 3 | mM | 4 |
| T4 G32 SSB | 312.5 μM | | 0.3 | μg/μl | 1.2 |
| | (10 μg/μl) | | | | |
| T4 UvsX | 43.5 μM | | 0.12 | μg/μl | 2.4 |
| | (2 μg/μl) | | | | |
| T4 UvsY | 71.4 μM | | 0.03 | μg/μl | 0.6 |
| | (2 μg/μl) | | | | |
| Bsu | 5 | U/μl | 5 | U | 1 |
| Creatine Kinase | 2 | μg/μl | 0.1 | μg/μl | 2 |

CRISPR mix

| 2.1 NEBuffer | 10X | | 2.1 | 10X |
|---|---|---|---|---|
| | | | NEBuffer | 20 μM |
| LbCas12a | 20 | μM | LbCas12a | |
| HPV16 crRNA-v2 | 100 | μM | HPV16 | 100 μM |
| | | | crRNA-v2 | |
| ssHEX | 100 | μM | ssHEX | 100 μM |

| | | | |
|---|---|---|---|
| glycerol. | 100% | glycerol. | 100% |
| DNA template | — | — | — |
| Total | | | 50 |

The methods of the subject invention relate to testing that using loop mediated isothermal amplification (LAMP) and CRISPR-based signal transduction for fluorescence visual readout, electrochemical-digital readout, or other colorimetric readouts. In certain embodiments, the LAMP forward inner primer or backward inner primer can comprise a PAM code downstream from the complementary region of the target sequence of the primer. In other embodiments, the loop structure containing LAMP primer can contain an about 20-unit long amplifier loop region, an about 7 base pair long PAM containing locker region, and/or an about 18-unit long target binding region. In certain embodiments, the amplifier loop region, the PAM containing locker region, and the target binding region are adjacent to each other in the LAMP primer.

In certain embodiments, the LAMP forward or backward inner primer with loop structure is designed with an introduced loop sequence that can be less prone to secondary structure. In some embodiments, the AapCas12b enzyme sgRNA is designed to recognize the complementary chain of the primer loop region after the initiation amplification of the sequence in the target RNA/DNA, which will become the loop region of the LAMP dumbbell structure that is crucial in the LAMP exponential amplification of the sequence in the loop region. In certain embodiments, the introduction of an extra sequence adjacent to the sequence complementary to the target RNA/DNA at the 3′ end in the introduced 20-unit long loop structure at the 5′ end of LAMP primer for CRISPR recognition can solve the sensitivity issue in one-tube reaction by protecting virus RNA/DNA targets in the initial amplification process from cis cleavage by CRISPR/AapCas12b.

In certain embodiments, the PAM sequence can be added onto a loop structure containing inner primers by designing a PAM code in the locker region of a loop structure containing primer, which is about a 7 base pair stem region at both sides of 20-unit long loop structure. This can facilitate the unwinding of the dumbbell structure amplicons to ensure the extension of a Bst DNA polymerase from the DNA 3′ end, such as, for example, Bst 2.0 polymerase (New England BioLabs, Ipswich, MA), and WarmStart Bst polymerase.

In certain embodiments, the binding sequence for sgRNA recognition is in the loop region of the LAMP dumbbell amplicon. In certain embodiments, the signal transduction by CRISPR/Cas unspecific collateral cleavage via trans cleavage activity is performed and can last more than 2 hours after trans cleavage activation by cis cleavage activity due to the function of the CRISPR/Cas enzyme towards the activator loop sequence in the LAMP amplicons.

In certain embodiments, the ssFQ reporter is about a 5 base long single stranded DNA sequence in reaction mix buffer with a fluorophore at the 3′ end and a quencher at the 5′ end, respectively, preferably with a TTTTT sequence in the middle. In other embodiments, the fluorescence signal is generated from excitation of cleaved fluorophore labelled ssDNA reporters. Further, the simple visual readout is realized by an optical filter on the top of the dark chamber to filtrate the extra lights with a wavelength other than that of the emission light (520 nm to 530 nm) to distinguish positive signal (green color) and negative (dark background color).

In certain embodiments, the CRISPR/Cas recognition sequence can be placed partially on the forward or backward RNA primer, such as about 30 to about 35 nts, by putting a PAM code downstream from the target for CRISPR/Cas recognition of the activator sequence, including at least about 10 nt downstream or upstream from the primer.

In certain embodiments, the high sensitivity of the reaction is achieved by adding 7.5% glycine in the reaction reagent mix without sacrificing amplicon-free performance.

In certain embodiments, the signal is generated from the single-stranded DNA fluorescence reporter (e.g., ssFQ) by separating the fluorophore and quencher to stimulate the fluorescence and generate a fluorescence visual signal. In other embodiments, the signal is generated from double or single-stranded DNA using a methylene blue reporter (dsMB/ssMB) by a differential pulse voltammetry (DPV) measurement to generate an immobilization-free electrochemical based signal. The immobilization-free electrochemical signal can be generated from the difference of diffusion coefficients between the uncleaved and the cleaved methylene blue labeled fragments of the DNA reporter, which can be identified by a differential pulse voltammetry (DPV) measurement using a commercial electrochemical platform.

In certain embodiments, the colorimetric signal is generated from the cleavage of the thiolated single stranded DNA (ssDNA) immobilized on a metal nanoparticle, such as, for example, a gold nanoparticle (AuNP), surface. The cleavage of ssDNA by CRISPR trans activity leads to aggregation of NPs with the AuNPs plasmonic surface resonance shifts according to the change of the dispersive states of AuNPs. In certain embodiments, the AuNPs plasmonic surface resonance shifts lead to a colorimetric change due to optical properties.

In certain embodiments, the rationally designed dual label at the 3′ end and the 5′ end of CRISPR trans cleavage reporter and the cleaved and uncleaved dual labeled reporter fragment can be used to lead the lined NPs to different locations on the lateral flow chip in a lateral flow readout. In certain embodiments, there are some capture materials modified on the lateral flow chip surface so that the cleaved reporter with certain NPs will be captured at a specific location on the lateral flow chip to enable a colorimetric readout. In certain embodiments, the on-chip detection is realized by designing the extraction-reaction-readout one-pot device utilizing magnetic beads to concentrate and transfer amplified sequences. In certain embodiments, the one-pot device includes the “3 in 1” chip where the extraction and reaction chambers are isolated by a mineral oil chamber. In certain embodiments, the sample concentration can use carboxylate-modified magnetic beads (the carbohydrate groups on the silicon surface with a Fe₃O₄ metal core are modified), where, in some embodiments, the FAM signal enhancement is specific to the fluorophore at the end of the reporter and is from carboxylate-modified magnetic beads with an SiO₂ surface. This embodiment can also comprise mineral oil film in the isolation between the extraction chamber and the reaction chamber. This embodiment can also comprise a commercial extraction buffer to lyse the virus and extract the RNA or DNA, pathogen onto the magnetic beads inside the extraction chamber that is isolated by the oil film without lowering the target concentration from about 500 to about 10⁶ cps/mL on the magnetic beads.

In certain embodiments, the AapCas12b enzyme sgRNA is designed to recognize the sequence before PAM code in this segment of the target that will become the loop region of the LAMP dumbbell structure crucial for LAMP exponential amplification.

In certain embodiments, the starting material is obtained via whole blood, plasma, serum, lymph, urine, saliva, tears, nasopharyngeal secretions, or any combination thereof. In preferred embodiments, the sample is obtained from nasal secretions or saliva. Once a swap is taken, nucleic acid material, including, for example, the viral RNA can optionally be isolated.

In certain embodiments, the CRISPR recognition sequence can be the pair chain upstream from the RPA primer sequence containing a PAM code while it is also necessary for exponential amplification.

In certain embodiments, the amplicon-free detection short product quantification analysis can be in both positive and negative samples using qPCR-based assessment. The sequence of qPCR primers can be designated based on the viral RNA and loop region sequence. The amplicon-free detection short product sequence characterization analysis in both positive and negative samples is using a confirm CRISPR/Cas added at the end of the reaction.

### Materials and Methods

### Point-of-Care Testing

Point-of-care testing encompasses anu tests that are performed at or near a patient and at the site where care or treatment is being conducted. The experiments of the subject invention focused on point-of-care tests that are accurate and financially available. An amplicon-free CRISPR-based one-pot loop-mediated isothermal amplification for point-of-care diagnosis of SARS-CoV-2 RNA. AapCas12b sgRNA can be designed, according to the subject method, to recognize the activator sequence. In this embodiment, the CRISPR activator is the loop region of the LAMP product, which is crucial for exponential amplification. During the reaction, amplicons would be cleaved by cis cleavage activity of AapCas12b enzyme till its activation after few-minute target amplification.

In this certain embodiment, the design of the subject invention can solve the contamination issue for point-of-care diagnostics by destroying the amplicons loop sequence at the end of each trail that can be hybridized and amplified again by LAMP primer without the virus RNA target. This can help stop false positive results in the non-lab area.

### Loop-Mediated Isothermal Amplification (LAMP)

The subject invention pertains to methods for contamination-free diagnostics in non-lab environments by introducing specific sequence in LAMP primers for AapCas12b sgRNA recognition as both an activation step and an amplified product cleaning step. In preferred embodiments of the subject invention, there are no amplicons left in the one-pot reaction tube ready for LAMP amplification since the region for loop primer binding is all destroyed by cis cleavage during the AapCas12b activation step.

In some embodiments, an amplicon-free CRISPR-based one-pot loop-mediated isothermal amplification for point-of-care diagnosis of SARS-CoV-2 RNA. AapCas12b sgRNA is designed to recognize the activator sequence. In this embodiment, the CRISPR activator is the loop region of the LAMP product, which is crucial for exponential amplification. During the reaction, amplicons would be cleaved by cis cleavage activity of AapCas12b enzyme till its activation after few-minute target amplification.

In this specific embodiment, the design of the subject invention can solve the contamination issue for point-of-care diagnostics by destroying the amplicons loop sequence at the end of each trail that can be hybridized and amplified again by LAMP primer without the virus RNA target. This can help stop false positive results in the non-lab area.

### CRISPR-Based One-Pot Detection Mechanism

In some embodiments, an amplicon-free CRISPR-based one-pot loop-mediated isothermal amplification for point-of-care diagnosis of SARS-CoV-2 RNA. AapCas12b sgRNA is designed to recognize the activator sequence. In this embodiment, the CRISPR activator is the loop region of the LAMP product, which is crucial for exponential amplification. During the reaction, amplicons would be cleaved by cis cleavage activity of AapCas12b enzyme till its activation after few-minute target amplification. According to our amplicon-free mechanism, the inner primer is designed by siting a PAM code downstream the target sequence. AapCas12b enzyme sgRNA will be designed for rapid recognition of the sequence before PAM code in this segment, which will be crucial for LAMP exponential amplification based on the LAMP dumbbell structure created in the initial stage. In this way, once there is a target viral pathogen, the target amplification reaction will be transduced to CRISPR signal amplification, amplicons will also be destroyed during this CRISPR activation process before there are too many amplicons (around 10⁹-fold amplicons at the end of traditional LAMP reaction). Amplicon-free CRISPR-based one-pot detection mechanism also has good compatibility with loop structure containing LAMP primer pool.

### Contamination Free CoLAMP Reaction

The reaction mechanism of contamination-free CoLAMP reaction by siting cis cleavage into loop sequence region in LAMP inner primers. Using this design, the CRISPR activation process is then by recognizing LAMP loop structure containing amplicons, without losing any RNA target templates in this step.

The sgRNA of Confirm-CRISPR/Cas enzyme is designed to check whether there is a sequence from LAMP amplification of a target viral pathogen in CoLAMP product using loop structure containing primers in case of the false positive signal from the introduced sequence on primer loop region primer if they are not well designed.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLES

A greater understanding of the present invention and of its many advantages may be had from the following examples, given by way of illustration. The following examples are illustrative of some of the methods, applications, embodiments, and variants of the present invention. They are not to be considered as limiting the invention. Numerous changes and modifications can be made with respect to the invention.

### Example 1—Amplicon Contamination-Free Point-of-Care SARS-COV-2 Diagnostics Using Fluorescence-Based Visual Readout

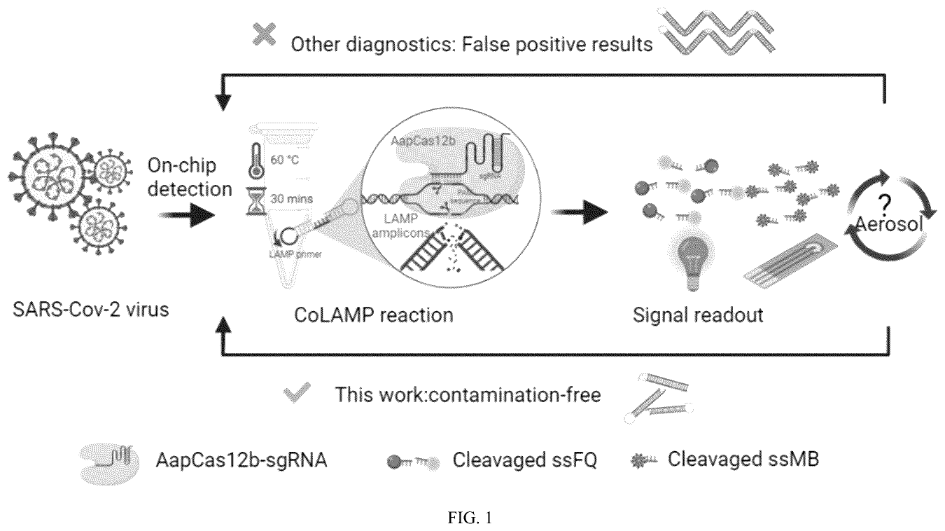

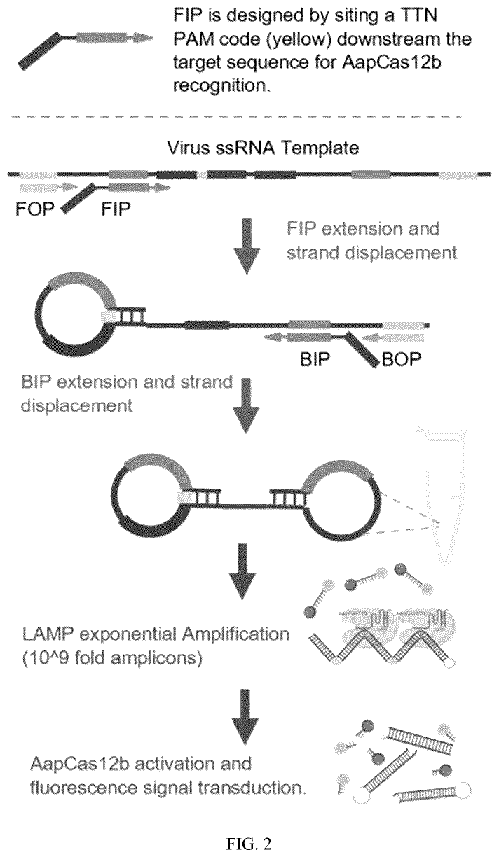

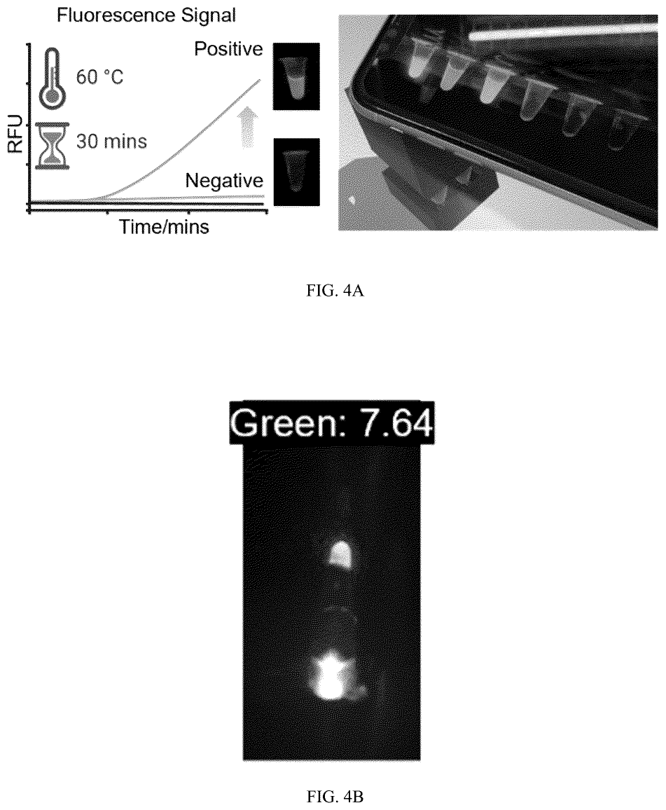

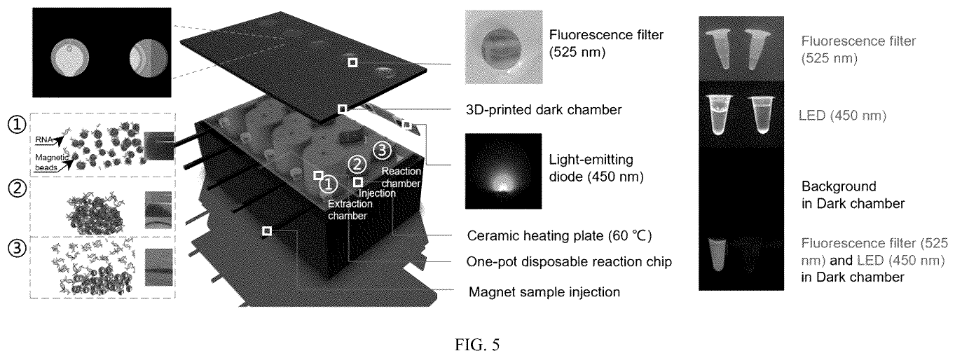

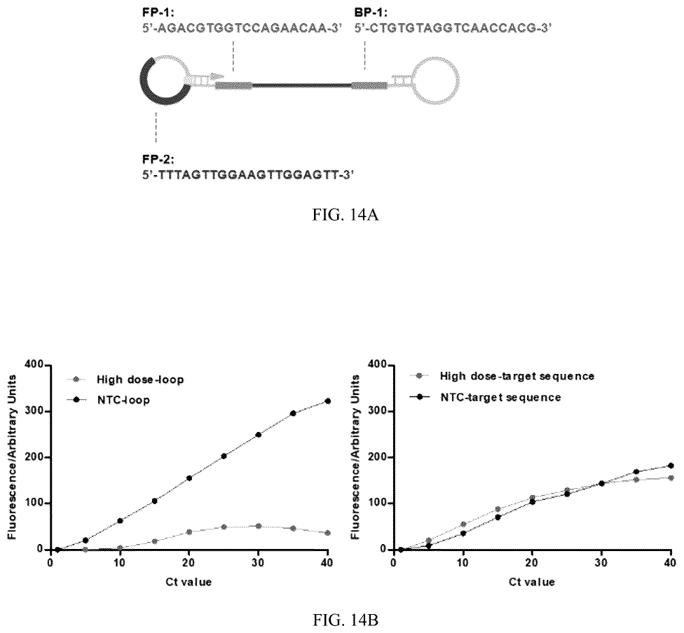

In each reaction, by designing the cis cleavage site into the loop amplification region in LAMP amplicons, AapCas12b can be initiated by double-stranded LAMP product and destroyed by specific cis cleavage activity, and then create nicks in other regions in dsDNA amplicons as well as cut the surrounding ssDNA fluorescence reporters and primers by non-canonical trans cleavage activity, through which we may observe contamination-free, rapid, visual detection (FIG. 1). As a proof-of-concept, we show that using this strategy, we can detect up to 0.5 copies/mL of SARS-CoV-2 pseudo virus in one-pot from extraction to signal readout in a nasopharyngeal swab background of a negative clinical sample (FIG. 4B). We also built up a simple visual readout prototype using a specific wavelength light source and a narrow wavelength filter suitable for different fluorophores, indicating large potential for on-site testing application even like home-base screening (FIG. 5). Moreover, we established stable figure treatment to transduce the visual readout into exported reports for smart phone users and can be further analyzed quantitatively (FIG. 8). Also, qPCR can realize product quantification analysis on loop and target region of short products in both positive and negative samples when dealing with high-dose samples (FIG. 14B).

### Example 2—Sensitivity Enhancement without Sacrificing Amplicon-Free Performance Utilizing Glycerol Additive in Crispr Phase

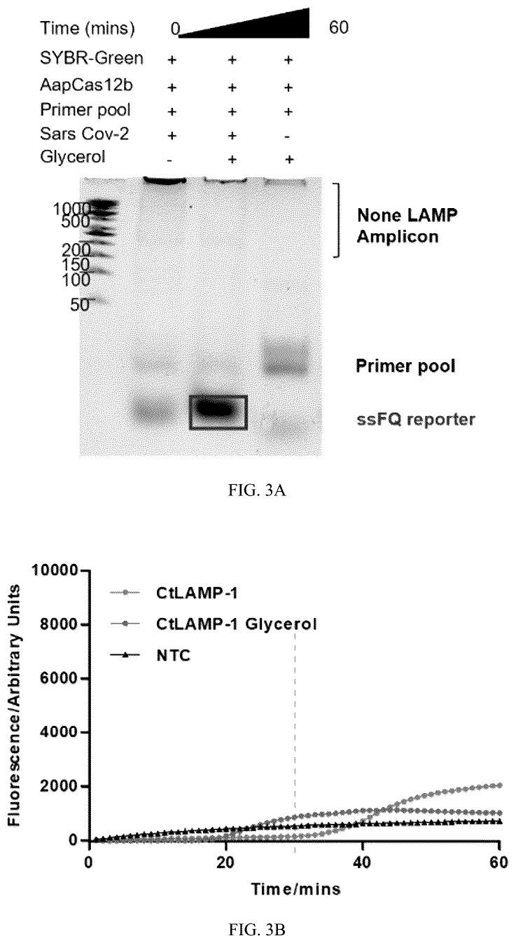

Sensitivity enhancement for at-home testing can be achieved by altering the viscosity physical property of glycerol to create a two-phase interface without interference on amplicon-free performance (FIG. 3A). Since glycerol exerted no positive impact on either the LAMP or CRISPR reaction system, the notable promotion in detection efficiency of the one-pot system may be on more account of its physical property (FIG. 3B).

### Example 3—Amplicon-Free Point-of-Care Diagnostics Using Nanoparticles (NPS) Based Colorimetric Readout

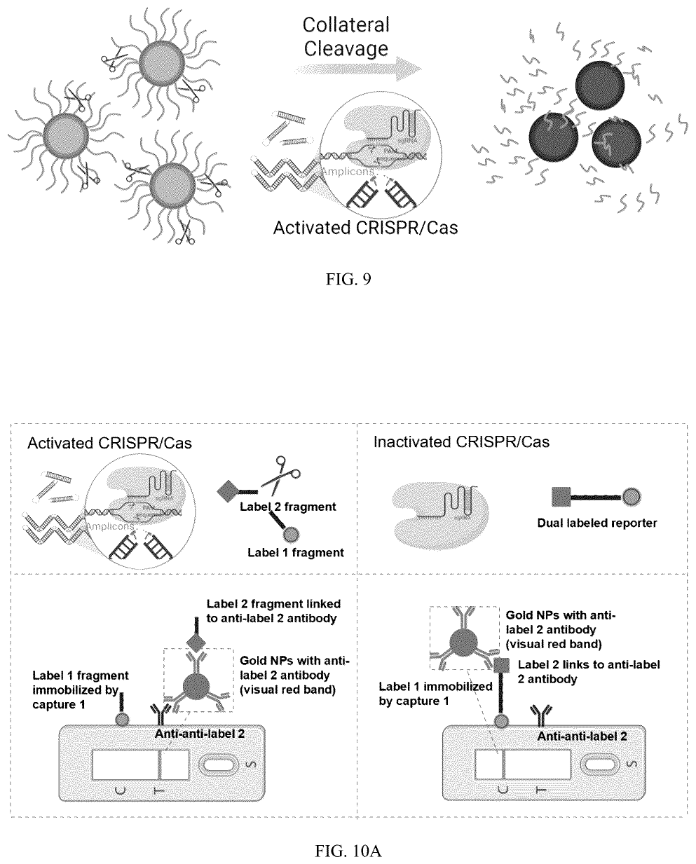

Based on the phenomenon that the optical properties exhibited by the dispersive states of metal, NPs, such as, for example, AuNPs can be easily identified with the naked eye or with UV lights, AuNP probes can serve as a fluorescence reporter. The thiolated ssDNA on the AuNP surface can be cleaved by CRISPR trans activity leading to aggregation of NPs (FIG. 9). Then the solution color may change from red to purple because the AuNPs plasmonic surface resonance shifts according to the change of the dispersive states of AuNPs.

### Example 4—Amplicon-Free Point-of-Care Diagnostics Using Lateral Flow Assay

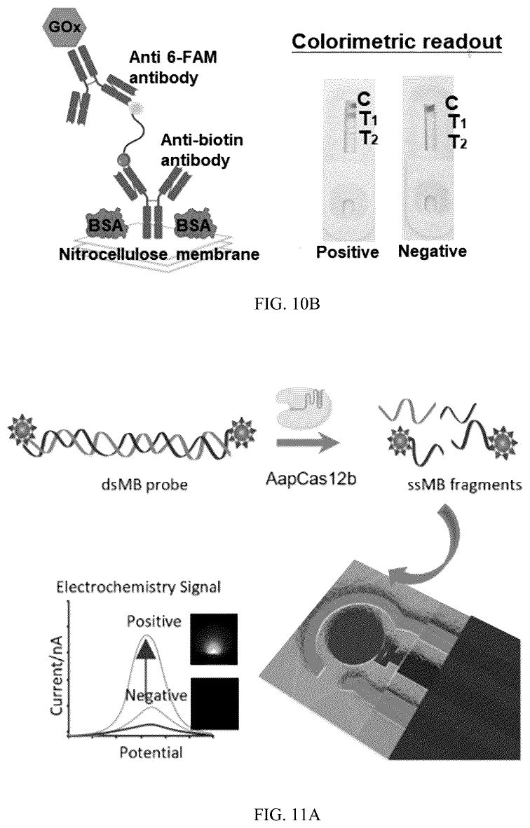

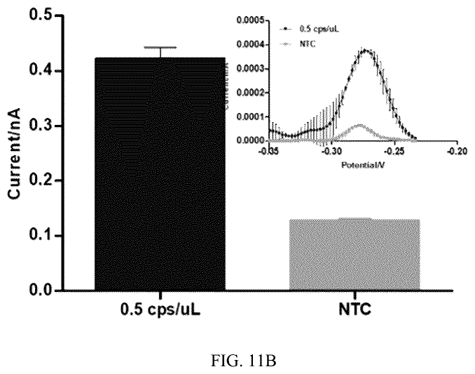

The amplicon contamination issue is usually more serious in an ending point assay, such as, for example, lateral flow, that there is a need to open the reaction tube to drip out the reaction solution onto sensor surface. Our amplicon-free mechanism can help with rational designing the dual label at 3′ and 5′ end of CRISPR trans cleavage reporter suitable for lateral flow immobilization-based assay. The signal reporter can be labelled with fluorophore, gold, or latex. In our amplicon-free CRISPR-based reaction, the cleaved and uncleaved dual labeled reporter fragment can lead the lined NPs to different locations on the lateral flow chip (FIG. 10A). Further, the metal nanoparticles that can show color band with naked eyes due to its plasmonic surface resonance. Alternatively, the NPs can be replaced by other colorimetric initiators, such as glucose oxidase, to trigger a downstream colorimetric readout (FIG. 10B).

### Example 5—Amplicon-Free Point-of-Care SARS-COV-2 Diagnostics Using Disposable Electrochemistry-Based Digital Readout

Instead of fluorophore, when using ssDNA, methylene blue (MB) labelled reporters (our detection platform) can be used as a disposable electrochemical testing card (FIG. 11A). Based on the immobilization-free electrochemical-based fundamental principle, the large difference in diffusion coefficients with different ssDNA lengths will result in large current signal-on performance during differential pulse voltammetry (DPV) scan measurement. The length difference from reporter with a longer length, such as, for example, 30 nts, to less than 10 nts fragments will be transduced to a signal-on current peak at a certain potential corresponding to methylene blue (MB) electrochemical label to distinguish positive and negative samples (FIG. 11B). Moreover, we established a blue teeth link in our testing card so that this digital readout can be exported reports for smart phone users.

### Example 6—Amplicon-Free Point-of-Care Diagnostics Extraction-Reaction-Readout One-Pot Chip Realized by Magnetic Beads

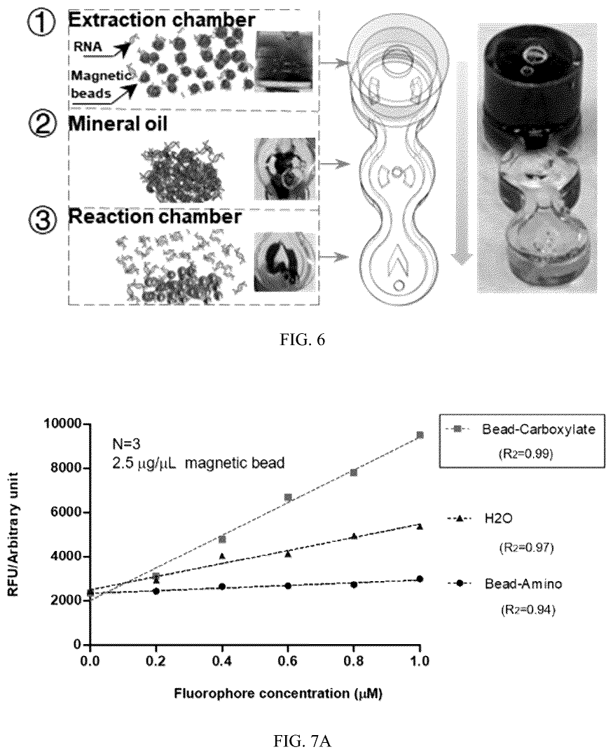

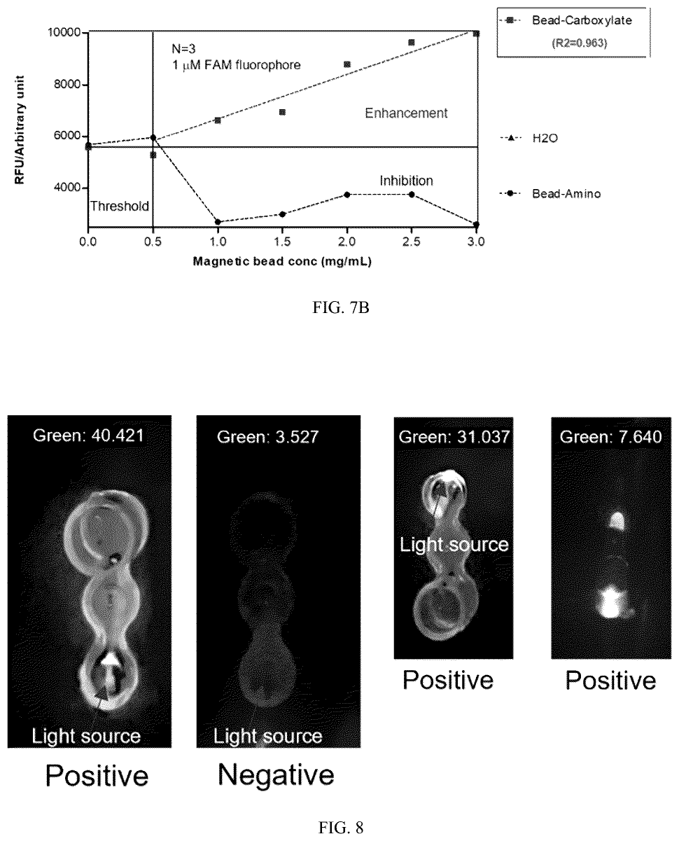

The sampling flow chamber automatic sample concentration and injection design is realized by magnetic beads moving sample from extraction buffer to reaction buffer isolated by mineral oil (FIG. 6). For fluorescence readout, carboxylate magnetic beads are used to avoid of inhibition effect of fluorescence signal because the excitation wavelength overlap with Fe₃O₄ metal core is removed to some extend by SiO₂ surface. (FIG. 7B).

### Example 7—Amplicon-Free Crispr-Based One-Pot Detection with Loop Structure Containing Lamp Primer Pool

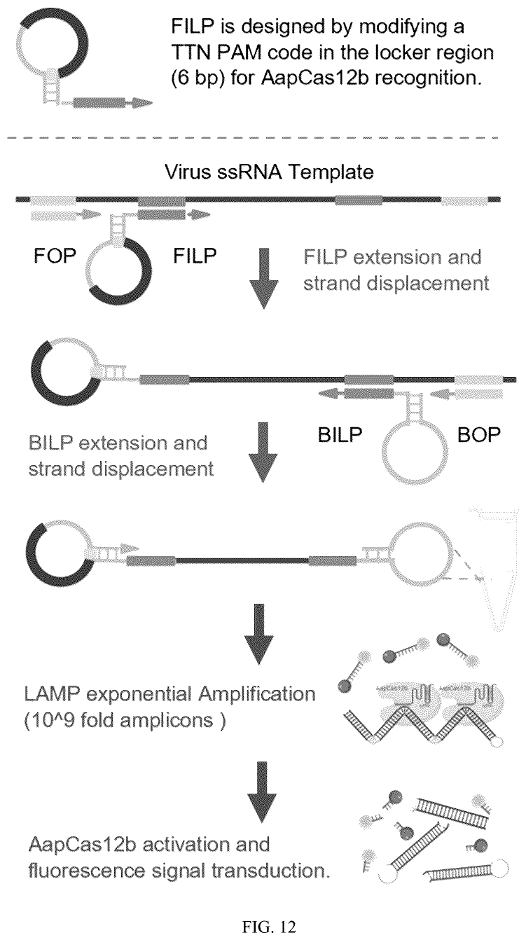

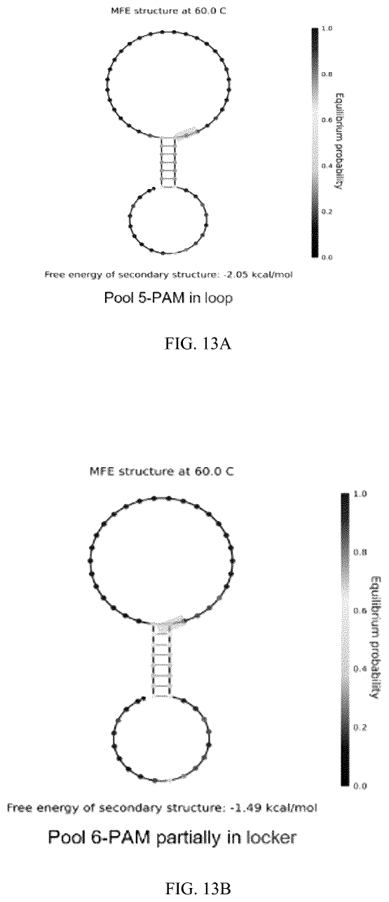

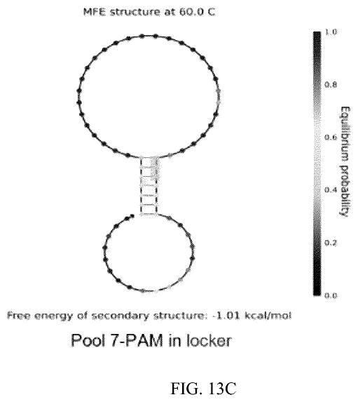

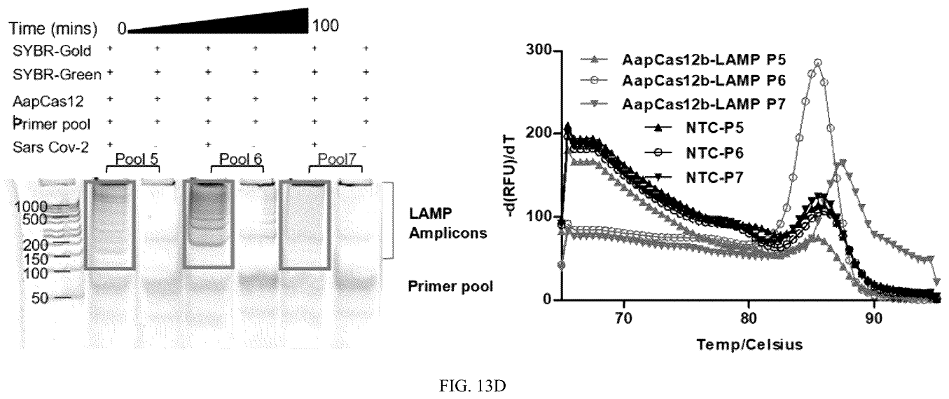

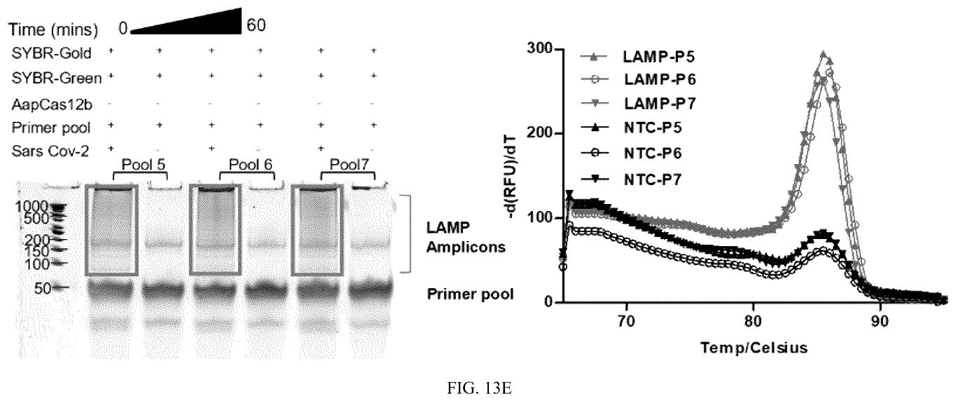

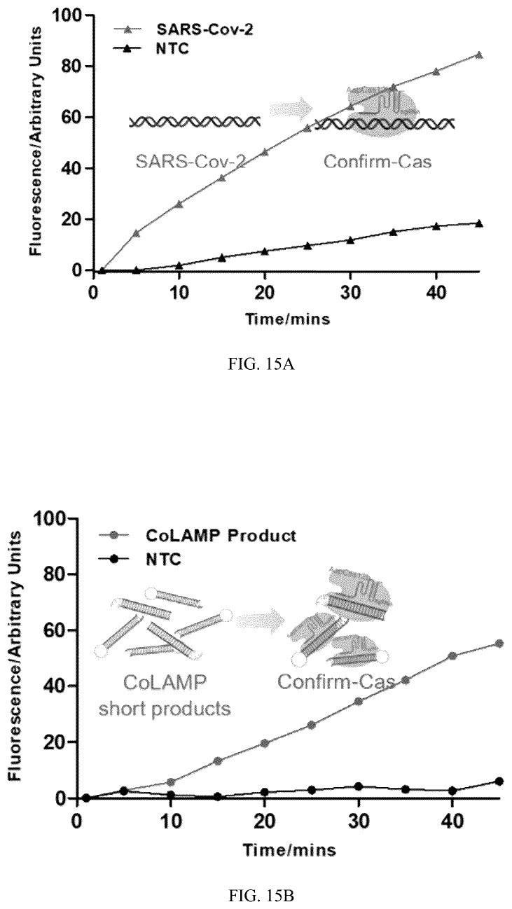

Since LAMP primer is not easily designed because of its unspecific amplification and secondary structure of the large loop structure (usually around 50 nts), we came up with novel design of LAMP inner primers that contain a smaller loop (30 nts) before the reaction (FIG. 12). This is similar to the primer pool in traditional LAMP, but the PAM code is designed within locker region (8 bp) of the loop-containing LAMP inner primers instead (FIG. 13C). CRISPR enzyme sgRNA will be designed for rapid recognition of the sequence in the loop region of the inner primer, which is crucial for LAMP exponential amplification, based on the LAMP dumbbell structure created in initial stage. In this way, once viral pathogen is targeted, the target amplification reaction will be transduced to CRISPR signal amplification, amplicons will also be destroyed during this CRISPR activation process before there are too many amplicons. In addition, the sgRNA of a Confirm-CRISPR/Cas enzyme can be designed to check whether there is sequence from LAMP amplification of a target viral pathogen in CoLAMP product using loop structure containing primers in case of false positive signal from the introduced sequence on primer loop region primer if they are not well designed (FIG. 15B).

### Example 8—Amplicon-Free Point-of-Care Diagnostics Initiated by Other Typical Isothermal Amplification Method, Such as RPA

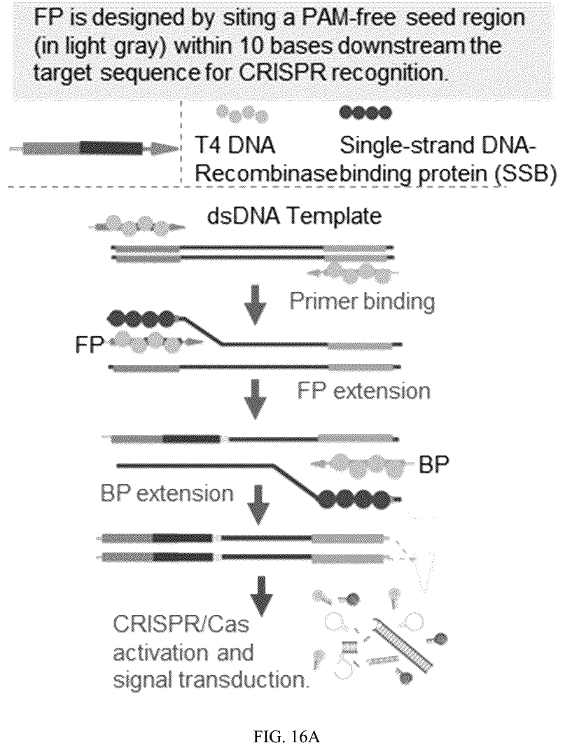

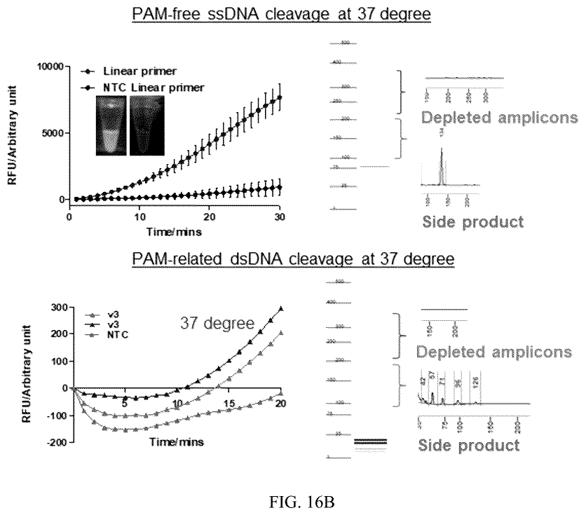

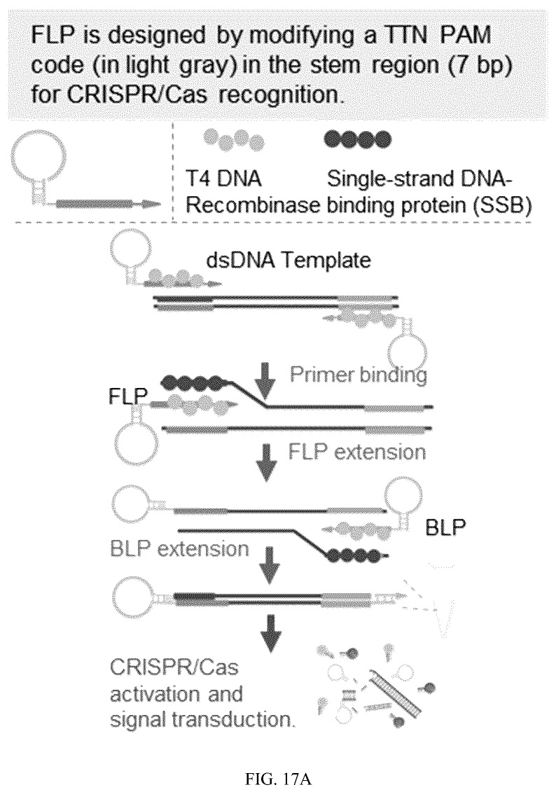

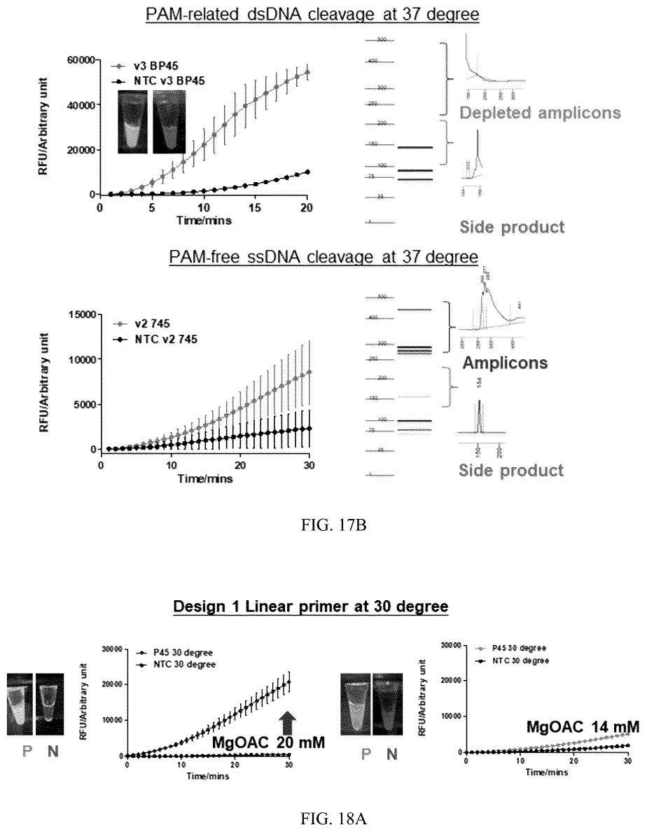

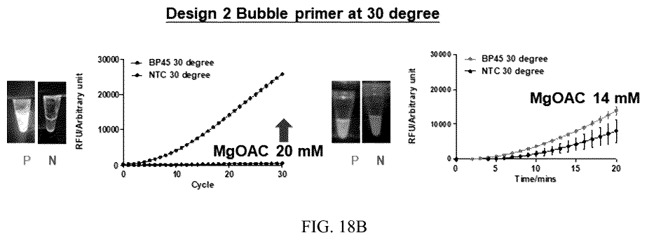

The amplicon-free mechanism can also be realized by other isothermal amplification by combining other CRISPR/Cas enzymes, such as, for example, Cas12a, by rational design of CRISPR guide RNA recognition site on the primer sequence that is exponentially amplified after the target binding initiation stage. The recognition site is supposed to be different than the primer binding site; thus, complementary to the target (30-35 nts for RPA) to avoid target waste at the initial stage of the amplification. It can be the sequence partially on the forward or backward primer by siting a PAM code downstream the target for CRISPR/Cas recognition. The primer sequence crucial for exponential amplification can be destroyed in CRISPR activation process before there are too many amplicons (FIGS. 16A-16B). Also, it should be the pair chain upstream the primer sequence containing a PAM code (FIGS. 17A-17B). Amplicon-free CRISPR-based one-pot detection mechanism have good compatibility with RPA by introducing extra sequence upstream the forward primer and backward primer for CRISPR/Cas recognition while it is also crucial for exponential amplification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Iliescu, F. S. et al. Point-of-Care Testing & mdash; The Key in the Battle against SARS-CoV-2 Pandemic. Micromachines 2021, Vol. 12, Page 1464 12, 1464 (2021).
2. Arons, M. M. et al. Presymptomatic SARS-CoV-2 Infections and Transmission in a Skilled Nursing Facility. N. Engl. J. Med. (2020) doi:10.1056/nejmoa2008457.
3. Wu, C. et al. CRISPR-Cas12a-Empowered Electrochemical Biosensor for Rapid and Ultrasensitive Detection of SARS-CoV-2 Delta Variant. Nano-Micro Lett. 14, (2022).
4. Budd, J. et al. Digital technologies in the public-health response to COVID-19. Nat. Med. 2020 268 26, 1183-1192 (2020).
5. Teixeira, W. et al. An all-in-one point-of-care testing device for multiplexed detection of respiratory infections. Biosens. Bioelectron. 213, 114454 (2022).
6. Mlcochova, P. et al. Combined Point-of-Care Nucleic Acid and Antibody Testing for SARS-CoV-2 following Emergence of D614G Spike Variant. Cell Reports Med. 1, 100099 (2020).
7. Benda, A. et al. COVID-19 Testing and Diagnostics: A Review of Commercialized Technologies for Cost, Convenience and Quality of Tests. Sensors 2021, Vol. 21, Page 6581 21, 6581 (2021).
8. Sawicki, R., Korona-Glowniak, I., Boguszewska, A., Stec, A. & Polz-Dacewicz, M. Sample pooling as a strategy for community monitoring for SARS-CoV-2. Sci. Reports 2021 111 11, 1-8 (2021).
9. Marshall, J. C. et al. A minimal common outcome measure set for COVID-19 clinical research. Lancet Infect. Dis. 20, e192-e197 (2020).
10. Yin, L., Man, S., Ye, S., Liu, G. & Ma, L. CRISPR-Cas based virus detection: Recent advances and perspectives. Biosens. Bioelectron. 193, 113541 (2021).
11. Joung, J. et al. Detection of SARS-CoV-2 with SHERLOCK One-Pot Testing. N. Engl. J. Med. NEJMc2026172 (2020) doi:10.1056/NEJMc2026172.
12. Chen, J. S. et al. CRISPR-Cas12a target binding unleashes indiscriminate single-stranded DNase activity. Science (80-.). (2018) doi:10.1126/science.aar6245.
13. Teng, F. et al. CDetection: CRISPR-Cas12b-based DNA detection with sub-attomolar sensitivity and single-base specificity. Genome Biol. 20, 1-7 (2019).
14. Ma, C. et al. A novel method to control carryover contamination in isothermal nucleic acid amplification. Chem. Commun. 53, 10696-10699 (2017).
15. Chandrasekaran, S. S. et al. Rapid detection of SARS-CoV-2 RNA in saliva via Cas13. Nat. Biomed. Eng. 2022 68 6, 944-956 (2022).
16. Liu, H., Thi Dao, T. N., Koo, B., Jang, Y. O. & Shin, Y. Trends and challenges of nanotechnology in self-test at home. TrAC Trends Anal. Chem. 144, 116438 (2021).

We claim:

1. A method of loop mediated isothermal amplification (LAMP) identifying of a nucleic acid sequence in a sample, the method comprising:
a) providing a sample containing a target nucleic acid sequence containing a first target region and a second target region;
b) hybridizing a forward inner LAMP primer to a first target region, wherein the forward inner LAMP primer comprises a complementary sequence to the first target region and a PAM code sequence downstream of the complementary sequence;
c) extending the forward inner LAMP primer to yield a displaced sequence, wherein the displaced sequence contains the second target region;
d) hybridizing a backward inner LAMP primer to the second target region of the displaced sequence;
e) extending the backward inner LAMP primer to yield a dumbbell sequence containing a loop structure;
f) amplifying the dumbbell sequence by CRISPR/Cas activation that specifically recognizes first and second target regions using cis cleavage activity; and
g) cleaving the surrounding reporters by collateral cleavage activity right after activation.

2. The method of claim 1, wherein the PAM code sequence is immediately downstream of the complementary sequence or at least 1, 2, 3, 4, 5, 6, 7, 8, 9, or 10 nucleotides downstream of the complementary sequence.

3. The method of claim 1, wherein the forward inner LAMP primer further comprises an amplifier loop region.

4. The method of claim 1, wherein the sample is whole blood, plasma, serum, lymph, urine, saliva, tears, nasopharyngeal secretions, or any combination thereof.

5. The method of claim 1, wherein amplifying the dumbbell sequence uses a Bst DNA polymerase.

6. The method of claim 1, wherein the dumbbell sequence contains a binding sequence for single guide RNA (sgRNA) recognition for CRISPR/Cas nucleic acid cleavage.

7. The method of claim 6, further comprising:
h) cleaving the amplified dumbbell sequence using CRISPR/Cas after activation by cis cleavage.

8. The method of claim 6, wherein the sgRNA targets a sequence adjacent to or at least 1, 2, 3, 4, 5, 6, 7, 8, 9, or 10 nucleotides upstream or downstream of the PAM code sequence.

9. The method of claim 1, wherein the sample contains a pathogen with genetic materials.

10. The method of claim 1, wherein the target nucleic acid sequence is single-stranded RNA, single-stranded DNA, or double-stranded DNA.

11. The method of claim 1, further comprising adding 7.5% glycine to the dumbbell sequence.

12. The method of claim 6, further comprising:
h) contacting a CRISPR/Cas sgRNA and a reporter to the amplified dumbbell sequence, wherein the CRISPR/Cas sgRNA comprises a target binding sequence specific to the amplified dumbbell sequence.

13. The method of claim 12, wherein the reporter is a single-stranded or a double-stranded DNA fluorescent reporter.

14. The method of claim 13, wherein the single-stranded or a double-stranded DNA fluorescent reporter is labeled at the 3′ end with a fluorophore and at the 5′ end with a quencher and further comprises a TTTTT sequence between the fluorophore and the quencher.

15. The method of claim 12, wherein the reporter is a single-stranded or a double-stranded DNA electrochemical reporter.

16. The method of claim 15, wherein the single-stranded or a double-stranded DNA electrochemical reporter is labeled at the 3′ end and 5′ end with a methylene blue label and further comprises a 30 to 60 bases -TTTTT- sequence between the two labels.

17. The method of claim 12, further comprising cleaving the reporter to generate a transduced signal from excitation of the cleaved reporter after CRISPR/Cas activation.

18. The method of claim 17, further comprising detecting the reporter transduced signal using on-chip detection with a magnetic bead to concentrate and transfer the amplified dumbbell sequence.

19. The method of claim 17, further comprising detecting a colorimetric signal using a lateral flow on-chip detection with the reporter labelled with a colorimetric initiator reporters.

20. The method of claim 18, wherein the magnetic bead is a carboxylate-modified magnetic bead.

21. The method of claim 20, wherein the magnetic bead is in a mineral oil film.

22. The method of claim 19, wherein the colorimetric initiator is glucose oxidase.


SEQUENCE LISTING (34 sequences)

SEQ ID NO: 1 - DNA, 18 bp, synthetic construct
  source 1..18: mol_type = other DNA
gctgctgaggcttctaag

SEQ ID NO: 2 - DNA, 21 bp, synthetic construct
  source 1..21: mol_type = other DNA
gcgtcaatatgcttattcagc

SEQ ID NO: 3 - DNA, 22 bp, synthetic construct
  source 1..22: mol_type = other DNA
ccttgtctgattagttcctggt

SEQ ID NO: 4 - DNA, 18 bp, synthetic construct
  source 1..18: mol_type = other DNA
tggcatggaagtcacacc

SEQ ID NO: 5 - DNA, 40 bp, synthetic construct
  source 1..40: mol_type = other DNA
gcggccaatgtttgtaatcagtagacgtggtccagaacaa

SEQ ID NO: 6 - DNA, 39 bp, synthetic construct
  source 1..39: mol_type = other DNA
tcagcgttcttcggaatgtcgctgtgtaggtcaaccacg

SEQ ID NO: 7 - DNA, 62 bp, synthetic construct
  source 1..62: mol_type = other DNA
ggccttcaactccaacttccaactaaatacgcccatcgaaggccagacgtggtccagaacaa

SEQ ID NO: 8 - DNA, 62 bp, synthetic construct
  source 1..62: mol_type = other DNA
ggctcgggcccactcctactaaactcccactcactaaccgagccctgtgtaggtcaaccacg

SEQ ID NO: 9 - RNA, 111 nt, synthetic construct
  source 1..111: mol_type = other RNA
gtctagaggacagaatttttcaacgggtgtgccaatggccactttccaggtggcaaagcccgttgagcttctcaaatctgagaagtggcacggaatgtcgcgcattggcat

SEQ ID NO: 10 - RNA, 111 nt, synthetic construct
  source 1..111: mol_type = other RNA
gtctagaggacagaatttttcaacgggtgtgccaatggccactttccaggtggcaaagcccgttgagcttctcaaatctgagaagtggcacaactccaacttccaactaaa

SEQ ID NO: 11 - RNA, 111 nt, synthetic construct
  source 1..111: mol_type = other RNA
gtctagaggacagaatttttcaacgggtgtgccaatggccactttccaggtggcaaagcccgttgagcttctcaaatctgagaagtggcacagcgctgggggcaaattgtg

SEQ ID NO: 12 - DNA, 18 bp, synthetic construct
  source 1..18: mol_type = other DNA
agacgtggtccagaacaa

SEQ ID NO: 13 - DNA, 20 bp, synthetic construct
  source 1..20: mol_type = other DNA
tttagttggaagttggagtt

SEQ ID NO: 14 - DNA, 18 bp, synthetic construct
  source 1..18: mol_type = other DNA
ctgtgtaggtcaaccacg

SEQ ID NO: 15 - DNA, 60 bp, synthetic construct
  source 1..60: mol_type = other DNA
  misc_feature 1: note = methylene blue
  misc_feature 60: note = methylene blue
tttttaaaaaaaaaaaaaaaaaaaaaaaaaaaaaaaaaaaaaaaaaaaaaaaaaattttt

SEQ ID NO: 16 - DNA, 60 bp, synthetic construct
  source 1..60: mol_type = other DNA
  misc_feature 1: note = methylene blue
  misc_feature 60: note = methylene blue
aaaaattttttttttttttttttttttttttttttttttttttttttttttttttaaaaa

SEQ ID NO: 17 - DNA, 60 bp, synthetic construct
  source 1..60: mol_type = other DNA
  misc_feature 1: note = methylene blue
  misc_feature 60: note = methylene blue
tttttttttttttttttttttttttttttttttttttttttttttttttttttttttttt

SEQ ID NO: 18 - RNA, 1261 nt, SARS-COV-2
  source 1..1261: mol_type = genomic RNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgatgggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 19 - DNA, 28 bp, synthetic construct
  source 1..28: mol_type = other DNA
ccaaggtttacccaataatactgcgtct

SEQ ID NO: 20 - DNA, 27 bp, synthetic construct
  source 1..27: mol_type = other DNA
tctttcattttaccgtcaccaccacga

SEQ ID NO: 21 - RNA, 41 nt, synthetic construct
  source 1..41: mol_type = other RNA
taatttctactaagtgtagatattttaccgtcaccaccacg

SEQ ID NO: 22 - DNA, 269 bp, influenza viruses
  source 1..269: mol_type = genomic DNA
tagatgtttaaagatgagtcttctaaccgaggtcgaaacgtacgttctttctatcataccgtcaggccccctcaaagccgagattgcgcagagactggaaagtgtctttgcaggaaagaatacagaccttgaggctctcatggaatggctaaagacaagaccaatcttgtcacctttgactaagggaattttaggatttgtattcacgctcaccgtgcccagtgagcgaggactgcagcgtagacgctttgtccaaaatgccctgaatg

SEQ ID NO: 23 - DNA, 30 bp, synthetic construct
  source 1..30: mol_type = other DNA
accgaggtcgaaacgtatgttctctctatc

SEQ ID NO: 24 - DNA, 30 bp, synthetic construct
  source 1..30: mol_type = other DNA
tctacgctgcagtcctcgctcactgggcac

SEQ ID NO: 25 - RNA, 41 nt, synthetic construct
  source 1..41: mol_type = other RNA
taatttctactaagtgtagatagggggcctgacggtatgat

SEQ ID NO: 26 - DNA, 288 bp, synthetic construct
  source 1..288: mol_type = other DNA
tattatgtcctacatctgtgtttagcagcgacgaagtatcctctgctgaaattattaggcagcacttggccaaccactccgccgcgacccatcccaaagccgtcgccttgggcaccaaagaatcacagacgactatccagcgaccaagatcagagccagacaccggaaacccctgccacaccaataagttgttgcacagagactcagtggacagtgctccaattctcactgcagttaacagctcacacaaagcacggattaactgtaatagtaacactacacccatag

SEQ ID NO: 27 - DNA, 27 bp, synthetic construct
  source 1..27: mol_type = other DNA
aagtatcctctgctgaaattattaggc

SEQ ID NO: 28 - DNA, 28 bp, synthetic construct
  source 1..28: mol_type = other DNA
ctattacagttaatccgtgctttgtgtg

SEQ ID NO: 29 - DNA, 61 bp, synthetic construct
  source 1..61: mol_type = other DNA
agatttcgaaggtgtgagggtgggagagaaatctaagtatcctctgctgaaattattaggc

SEQ ID NO: 30 - DNA, 62 bp, synthetic construct
  source 1..62: mol_type = other DNA
agatttcgaaggtgtgagggtgggagagaaatctctattacagttaatccgtgctttgtgtg

SEQ ID NO: 31 - RNA, 41 nt, synthetic construct
  source 1..41: mol_type = other RNA
taatttctactaagtgtagatagaggatacttcgtcgctgc

SEQ ID NO: 32 - RNA, 41 nt, synthetic construct
  source 1..41: mol_type = other RNA
taatttctactaagtgtagatgccaagtgctgcctaataat

SEQ ID NO: 33 - RNA, 41 nt, synthetic construct
  source 1..41: mol_type = other RNA
taatttctactaagtgtagatcagttaatccgtgctttgtg

SEQ ID NO: 34 - RNA, 41 nt, synthetic construct
  source 1..41: mol_type = other RNA
taatttctactaagtgtagatgaagtatcctctgctgaaat